SEQUENCE LISTING (18 sequences)

SEQ ID NO: 1 - protein, 659 aa, Zea mays
  VARIANT (185): X at position 185 is an unknown residue.
MPLIRYYVDHVHVAVEARALLGRLEFQKGNVEVALRVFDGIDLQAAIQQFQPSLSDKTPSKKGRTKSEIPSSVPQNNPASLVLEAIYLKSLSLQKLGKSTEAANQCKSVLDSVESIFQNGAPDIEQKLQETVNKSVELLPEAWKHAGSNQEALASYRRALLSPWNLDDECRTRVQKRFASFLLYXTNIDWSPPSMAQQVEGCFVPKNSVEEAVLLLMVVLRSWYQGKTHWDPSVMEHLTYALSLCGEPLVLAKQLEEVLPGVYPRTERWATLALCYYVAGQKDVALNFLRKSLNKLENPNDVLALLLAAKICSKERHLASEGVEYARRVIACAESSDPHLKSVGLHFLGSCLGNKSKVVSSDYQRSLLQTETLKSFAESIGLNRYNADLIFDMGVEYAEQRNMNAALRCAKEFIEATGGSVSKGWRLLALILSAQQRFSEAEVATDAALDETAKLDQGSLLRVKAKLKVAQSSPMEAVEAYRALLALVQAQKNSPGGSCKSATEDADGSVSEFEIWQGLANLYSSLSYWRDAEICLNKARALKLYSAATLHAEGYMHEARDQTTDALAAYVNAFSTELEHVPSKVAIGAMLSKQGPRFLPAARCFLSDALRVEPTNRMAWLYLGKVHRSDGRISDAADCFQAAVMLEESDPVESFSSLS

SEQ ID NO: 2 - protein, 18 aa, Artificial Sequence
  Description of Artificial Sequence Synthetic peptide
VSKGWRLLALILSAQQRF

SEQ ID NO: 3 - DNA, 4978 bp, Arabidopsis thaliana
  misc_feature (1)..(4978): N indicates A, T, G or C.
tgccatgttatgagagggacacttatagagattttggtgccttagtataataccntatgtcctctgtgtgttcgatgtatagatgcaagcatgactctagcttagatactcacgtgaactggatgagggagtggaactaaggaactatgggaaatatcctagctgcaagttaggactttgatgtatcccctctaatgtattatggtcatgtttatgtgtgcatgtgtctatgtctatgtgttccaatgtttcttcatacataactctcctcctcctttttataccattaaggaggatagaattatcttcctgcttgcatggtggtacccatccaacggaggtttaactactcctaacttgtagttgtagtgctatatgttgggcacttatttttgactcctttgggtatcaaaagcaaggcaacataatgagattgaatctaaccttcatctttaaattcaccttctttgaggagaaggttggatttaaagatgaaggtgtgacaataattaataatgacaatgttaacaaaatagtaatttgaagaatatagatatgaacatcaaagatatcatgtaaacttatcaacattgcttacttccttcggttttttcagtatcaaatcacagattacatttataactttagctttgtgatgcttacaagacgaaggaagaattataacttcatagatgagagctattcgccctttatctgatcataagatgtccaaatacatggcactattcctttatttataagtacagagtacaactttgataaaattacatttacatcctcaaggttatacatttgagctatttagcatggcaagggtgaggacgtcttttcctatcaccgtcttgatgagcgagtccaagaaggcttgcttcgactttgtctggcttcaccctttgtccttttatgatagaatcccaaagcttgtttttagtctatgaccgattcgtccgaagatgctttttgaatgagtgcctgtaatacctagaaatgcactagaaaacaattgttatcttggtgttttgaggaccttcagaaggagaaggcccccaacactatacataatgcatgcctctcatacgctactatgttgaccatgtgcatgttgcagtggtaggtacatgagaccgtactatcgagtataatataattgctaggtgatgttgctacataccctttatagtgcttgagatacttctgttgtcctagcaatggctacaacggatgttgtactcgcacacataacatagtgtttatgggtacacctatagccccaagaacgacaccgatgatttgaaccataatgtcacccatcgcaacaaatatgatgtaaaggagcaccaacttatttgtcaagcaccatgtgccactttaatcactatagcatgtggcgctcgaatcatatcttctggtatcgtgcaatcctaggggcatctaagaaatatcatagaacctttgtagggcctgagaacctgaccgatttgatgcttgccctccgaggcagaggccatgggacctatccattttaaggcttggcctctaggcacaggaccccaggagtgctaggatggcttggcctctagggcgaaagccacatgacctgaccaatttggggtttagccctaggagtactacgacggctcttagtcgggtctaccgtgagtttatctcctcctaatggaatcctagagggcgctttgctagcatccgaggatatgccttaccttgagcactggaaataatgtacacatcttttgatacttatttggatatcaattttttttgcctttttcctttgattacgaataaatgggtcacaaaatctactatgcaaattcgtattcttgttttaagtattgagcttgcaaaattttattgttatttgtaataatatgtttgataacataagaaaagatcaacatcaaatttcatacatatctatttaaaatattaaatttgttttaacaattcggatatctactttcatccttattagatttaatactaggtgtgctccctaacctatgaagattctttagtgtaacctgttcaaaaaagattattcaatgtatcctctttttttntgctatgacgtttcttttggttcctgaggctgagtatatgcaaatttatatattaattatcaataggaagcaagagctctccttggaagactggaattccagaaaggaaatgtggaagttgctctacgtgtatttgacggaattgaccttcaagctgcaattcagcagttccagccatccctgtctgataagacaccatcgaagaaagggcggacaaaatcagaaataccaagttccgtcccgcaaaataatccggctagccttgttcttgaagctatttacttgaaatcactgtctcttcaaaagctagggaaatcgacaggtacaaatcactgcacacatatcattattggacgatttctttcttatatatattatgaactctttgatgactaattaatgcaaaaacctgttgaacaagcagaagctgctaaccagtgcaaaagtgtactcgattctgttgagagtattttccagaacggcgctcctgatatcgagcagaagttacaggaaactgtgaataaatccgttgaacttcttcctgaagcctggaaacacgctggctccaaccaggaagcattagcgtcataccgacgagctcttcttagtccatggaatcttgatgatgaatgtcggactagggttcaaaagagattcgcgtcttttctactgtacancaccaacatagattggagccctccaagcatggctcagcaagttgaaggctgttttgttccgaagaatagcgtggaagaggccgttctactcttgatggtagtgcttaggagctggtaccagggcaagacccattgggatccttcggtgatggaacacttaacctatgccctgtctctttgtggtgaaccactggttcttgcaaagcagcttgaagaggttttgcctggagtgtatcctcgcacggagagatgggccacactagcgctctgctactacgtagctggccagaaagatgtggcgctgaatttcctgaggaagtccttaaacaagcttgagaatccaaatgacgtcctcgccttattgttagctgctaagatatgtagcaaggagcgccatcttgcttctgagggcgtcgaatacgcaagaagagtaatcgcctgtgccgaatcatcggatcctcatctgaagagcgtcggcctccatttcttggggagctgtctaggcaacaagtccaaggtcgtttcgtcggactaccaaagatctctgctgcagacggagactctcaagtcgtttgcagagtcgattggccttaaccgctacaacgccgacctcatattcgacatgggagttgagtacgccgagcaaaggaacatgaatgccgccctgagatgcgccaaggagttcatcgaggcaacgggcgggtcggtttcgaaaggatggaggctgctagcgttgattctttctgcgcagcagaggttctcggaagcggaagtcgcgacggacgctgccttagatgagactgcgaagctggatcaaggatctttgcttagggtaaaggccaagttgaaggttgctcagtcgtcgcccatggaagcagttgaagcgtaccgtgctcttctggctctcgttcaggcccaaaagaactccccgggcggatcttgcaagagcgctacagaggtatatatgtttttttttgtttcaagggcaaaaggaacttcaaattctgccatggttgaatttttaagggctagtttgacaactctatttttctaaggaattcctatttttatgaggtaaaatgaactaatttctcttagtcgtaaaatggagttgccaaactaggcataaatttgtcttgtgaagttttcccattaattcactcactcttcacttattactattcaggacgcggatggtagtgtctccgagtttgaaatctggcaaggtcttgctaatttgtactccagcctctcgtactggagagatgctgagatttgtttgaacaaggctagagccctcaagttgtactcagctgcaacactccatgcagaaggtgaggcacatttcattacgtacccttgccctttatgcatgcatatgttagtgaccatgcatgcatgcatttctctttaacatgggcatttacatatatgtttattatttacatatagatgatactcgtccatatatgcaggctacatgcatgaggcgcgagatcaaacaacggatgcactggctgcctacgtcaacgcgttctcgaccgagcttgagcatgtgccgtccaaggtggccatcggcgcaatgctctccaagcaaggtcccaggtttctacctgcagcgaggtgcttcctctccgacgccctaagagtcgagcccacaaatcggatggcttggctttacctaggaaaagtgcacagatctgatgggaggatctctgacgctgccgactgcttccaggcagcggtgatgctagaggaatcggatccagtggaaagtttcagttcgctctcatgagagatgcagccagtacggcatgcttgcatgaagctcccatccctccatgtggggttggtttcttccttcccgctttatttttctcctgttgcatagttggaggaaattatgtttggcggcagatgtaggtgttgcaattatgcgaagctgcatttcattgctcaatttcgttgtataaataattgttgctcccatttttatggatactgtattacatgtaacatgcatgtgagactaatttcgtcaccagaaactactacagtataaatatccagagtgttactgatttcgacaattcttgctgtactttttaactgtgcttattttggttaaattatcgagtatgaccaccactctaagcctacagtgagatccgttgacctgcaggtcgacctcgagnangg

SEQ ID NO: 4 - DNA, 3984 bp, Arabidopsis thaliana
ttatagttatgaaacttaatgattagcgagttgttgtagtagcagcagcttaaggatacatgtatatatagttacatttttatgttcgttactatttttgatttgtgtcgatgttaattaatttcttactaagaaacgactcttaagtttcgagcaaaatcacataccgtaaatacttgtactttatttcggaaatatgatgcatccatatcaatctttatcgtataataagcagtgcctaatctaatctcttccatcctgaaaattaatgactttgtaagataggtgatatatgatatatcagaatattaatttgatgtgttaccaagcttgaagcattagtaagtaagtaggacacattttttttggatttgtttatttatttctagttaacaggtaagagatataggaagtgacaagtactatcttttatttgaaaaaatatattctctttgatgcaatgaaactaaacatttcacaactctattttgtggtcaatttatatggattacatgttccacacaatgtatgtaagttagttcactaaacgtaaaccttaagggtgaagcaatgcaaagaaggctttaaattgccagactgtgcgtaacatttacaatcatttgctgctttttaatcatagagctacgtatatatattgtatgtactttatttttgcgaatatacgtatattagtatatttaatgtatgtatgatatagtcaatctgtattcatgcaaagtgaagttcatttatttattgaccaacaaaaagtgaaacattttaaagcaaaccgcgtataagttcataactttttctaaacaaattcttggaacttaacaaacaaaaaaaacttttcccaaacaatttaaccatagtataaagtacgaagttacgtattaacaatttaataagtactagtaaatttgttttcgtattaaatttatttaccataaaaataaaatcacacgtacgtctccagctcccagcctttaaaatggaaaaaaaaactcacaacgcaatgatacttttatacttaaaatgcgacttatatacttaaaatgtatcgaggtgaacaagctagcaaaatgtatcgaggagacaaacaaaatgtatgtaaatgcgacttatctacttaaaatgtatcgaggatcggagaacaagttagcaaaaaacaaaagatgaatggcaacaaaagaaaataaccaaaacttttttttacaaaatatgtacctgcttaatttaacgttacattatttcttggaaaatatgtttcatcgttaattattatggaaaattaattataaaaaaagttgcgtgattttgatttgtgcatggataatgataccgccaaacttaaaacggacaaacaaaatgggatccatcttgtttgtttgtagtttgtactgctcaaagaaaattcaacgacgcgacgtcactactcctcacccacttggcagcctcggggtcaaagaaaaacatttgtgtattctgttcctttaaaagttaaaacactgttattgctagaatctccaaattaatacttatgctgttatgcagtatgagtcgagtgtctgacttgtatgagttagggcctagtatgaataaataaacattattaatattaagatagttgttttcgataattgtttgataaggatgccactaaactcatacctcttagcttatacgaattgacttaattagacattaatacattatatctatatattatctagatttataattgctaagccaataggtcaaggtcttgtctaataaatgcatgcacaactaattcagtcaataatgtacctgtataatactacaaataattcaagctaattgatctatattgaagaacaaataagtaatctatttcggatttagtcttatcatgtgtctaaataaacacataactcttaagtcttaatgatttatttttgatagatatcaattataattatacaattacaaatgatttgatgattgactatacgtaagaactaactttgataattttgaattgggacaaatcattgaaggccttacgtttaagctttagatgtttcccaacgccaaaggagaatgaaaaggacagaccatcagtgatttgagtactcaatcaacatatttattatgtactttgagttaattaattttctattaataacaaaaatcaagcttgcacatttcaatgtgataagtatatgaataataatccaagctaatttttaagaaaagaggaatattgaaagcttgcaaattattcgaatgctagaggtccttaccttgcatgcaccttttgtaacaattacctatgggtgtggggaaatctagctagctacatattttcaattatttttccctattaaattgagattattgttataaaagaaaatgcccaaacttaattttcggggtttaaaattttgtttaaaaataaataaaatataagaaaagaaagaaaagtataatttgggttaaggggtttgaatatgattgatttgaatcgtcgtcgaaatgtatacgtcacctaacgcttttgttgctatactagtatcattaagtggaaattttaaagtcattaaaactcttctcatttttgtatttctaaaagagtcttaaggggtttgaatatgatttaaattatcttacaagtgtaaatgccatctaacgcttttgttgttatactagtattatttagtaataagatgctaaagtcactcaaactccagaatcaataatactccaagctatacatattagaattttaaaatagtatgaacactttcgataataaaaataccaaacttatttgggacactaaataagtttgggccgaaaatatttaaaagcccaatttaaactaaaattcatttaggctcttctcttctactaccttcttctatcgagccacaccgaatgaaattagtgaaactgctattggcttgtgaattgtgtgtgatggcgttaaagcctcttttagttcgtaaccgatgaaatgacagtaatagccttgagaaacactgaaaattacagaaagagagtttgaactttgaagacaaaacaggtgtttctatttctctccccgttcacgttctgcaacatcggaagcacgtacggctcctaagactccgttttgcttctttttttttaaaacacattcttattataaatataaaaaaaacaaagagagatcaaaaacaaaaagtttcctctctttttctaattttttaaagtttctttcatcttcttcagatccgaattgtcgccgcgaaattcgtcagtgcagcttcttcttcttcgcgtactttattcgatcggctgtctgaagaacatgaagccgatgatcgtaggtacgttagattcatttttccgaaattggctttttgatttttctgatcgaaacgatgcgaggttcaatttcatcattgttttgaaatctatgacttacaaaagtaatggcgttgacagatttgttcttaataaggacccacatttttgctgaattttggaacaaacattgttcttctttgatttcaaaacaagaattagaaaattcatttatcatgtaatctatttagctgatgtgacgatgaacagatcaaaggaatgtagtctcgaattgtttaaggttataatgattcctctaagtgaaaaaaaaaaaaaaagcagaaaaaaaagttagaaagatgacaaagttgaagatttctttttgtctttgaagcttctatttttttggtgggtcctttttaagacaatgatttcaattcttggattttgtctgaagaaaaatgttgctgttcttctctttacaatgtttttgattgtgagcttgcgttgacttaaatcatgtcatatattttggtttctcacggttttatttattgtgccaagtgatgcagttgctgctagttacggtggattgatgtttggatggacgcagaaattttgatgtgggtttagtctaaaaggtgaagaaca

SEQ ID NO: 5 - protein, 23 aa, Artificial Sequence
  Description of Artificial Sequence Synthetic peptide
AKLDQGSLLRVKAKLKVAQSSPM

SEQ ID NO: 6 - protein, 23 aa, Artificial Sequence
  Description of Artificial Sequence Synthetic peptide
ISSKEMVRLKKLVAYWKEQAGKK

SEQ ID NO: 7 - DNA, 1980 bp, Zea mays
  misc_feature (554): N at position 554 is A, T, G or C.
atgcctctcatacgctactatgttgaccatgtgcatgttgcagtggaagcaagagctctccttggaagactggaattccagaaaggaaatgtggaagttgctctacgtgtatttgacggaattgaccttcaagctgcaattcagcagttccagccatccctgtctgataagacaccatcgaagaaagggcggacaaaatcagaaataccaagttccgtcccgcaaaataatccggctagccttgttcttgaagctatttacttgaaatcactgtctcttcaaaagctagggaaatcgacagaagctgctaaccagtgcaaaagtgtactcgattctgttgagagtattttccagaacggcgctcctgatatcgagcagaagttacaggaaactgtgaataaatccgttgaacttcttcctgaagcctggaaacacgctggctccaaccaggaagcattagcgtcataccgacgagctcttcttagtccatggaatcttgatgatgaatgtcggactagggttcaaaagagattcgcgtcttttctactgtacancaccaacatagattggagccctccaagcatggctcagcaagttgaaggctgttttgttccgaagaatagcgtggaagaggccgttctactcttgatggtagtgcttaggagctggtaccagggcaagacccattgggatccttcggtgatggaacacttaacctatgccctgtctctttgtggtgaaccactggttcttgcaaagcagcttgaagaggttttgcctggagtgtatcctcgcacggagagatgggccacactagcgctctgctactacgtagctggccagaaagatgtggcgctgaatttcctgaggaagtccttaaacaagcttgagaatccaaatgacgtcctcgccttattgttagctgctaagatatgtagcaaggagcgccatcttgcttctgagggcgtcgaatacgcaagaagagtaatcgcctgtgccgaatcatcggatcctcatctgaagagcgtcggcctccatttcttggggagctgtctaggcaacaagtccaaggtcgtttcgtcggactaccaaagatctctgctgcagacggagactctcaagtcgtttgcagagtcgattggccttaaccgctacaacgccgacctcatattcgacatgggagttgagtacgccgagcaaaggaacatgaatgccgccctgagatgcgccaaggagttcatcgaggcaacgggcgggtcggtttcgaaaggatggaggctgctagcgttgattctttctgcgcagcagaggttctcggaagcggaagtcgcgacggacgctgccttagatgagactgcgaagctggatcaaggatctttgcttagggtaaaggccaagttgaaggttgctcagtcgtcgcccatggaagcagttgaagcgtaccgtgctcttctggctctcgttcaggcccaaaagaactccccgggcggatcttgcaagagcgctacagaggacgcggatggtagtgtctccgagtttgaaatctggcaaggtcttgctaatttgtactccagcctctcgtactggagagatgctgagatttgtttgaacaaggctagagccctcaagttgtactcagctgcaacactccatgcagaaggctacatgcatgaggcgcgagatcaaacaacggatgcactggctgcctacgtcaacgcgttctcgaccgagcttgagcatgtgccgtccaaggtggccatcggcgcaatgctctccaagcaaggtcccaggtttctacctgcagcgaggtgcttcctctccgacgccctaagagtcgagcccacaaatcggatggcttggctttacctaggaaaagtgcacagatctgatgggaggatctctgacgctgccgactgcttccaggcagcggtgatgctagaggaatcggatccagtggaaagtttcagttcgctctcatga

SEQ ID NO: 8 - DNA, 2476 bp, Arabidopsis thaliana
atgctcgggaatcaatccgcggattttagtgagaagggggaagatgagatcgtcagacagctttgtgctaatgggatttgcatgaaaacaactgaagttgaagcaaagcttgatgaaggaaatattcaagaagctgaatcttctttgagagaaggattatctctcaatttcgaggtttgtttttgtggttttccaaactccattaaaccaacttttataagtaaatgattagattttatttgtttttgtggttacaggaagcaagagcacttcttggaagattggaataccaaagagggaatttagaaggcgcacttcgtgtctttgaaggtatcgaccttcaagcagctatccagcggttacaggtttccgtgcctcttgagaaaccggctactaagaaaaaccgtccccgtgaaccgcagcaatcagtttctcagcatgctgctaacttggtccttgaagctatctacttgaaagccaaatcccttcaaaagcttgggagaataactggtatcattccctttcacctttgaaaatgtgaatctgaggcattgatcttggtacagtctttaaatctaaatcttcccttgctgggttttggtcttgtagaggctgctcatgaatgcaagagtgttcttgattctgttgagaagatatttcagcaagggataccagatgctcaagtggataacaaacttcaagaaaccgttagccacgccgttgaactacttcctgcgctatggaaagaatctggtgattatcaagaagccatatctgcttatagacgcgcgcttttaagccaatggaatcttgataatgattgttgtgcaaggattcaaaaagattttgcagtctttcttttacattctggagtcgaagcgagtccaccgagtttaggttctcagatagagggatcgtacatacctagaaacaacatagaagaagccattcttcttctaatgattcttttaaagaagtttaacctcgggaaagcgaaatgggatccgtctgtgtttgagcaccttacctttgcgttatctttatgtagtcagaccgcggttctcgccaagcagcttgaagaagtaatgcctggtgtgtttagccgtattgagcgttggaacactttggctctttcttatagtgcagcaggtcaaaacagtgctgcagttaaccttcttagaaagtctctgcataaacacgaacaacccgatgatcttgtggcgcttttgttagctgctaagctttgcagtgaagagccttctttagctgctgaaggtacgggttatgcgcagagagcgataaacaatgctcaaggtatggatgagcatttgaaaggcgttggtttgaggatgttaggactttgtttagggaaacaagcgaaggttccgacatcggattttgaaagatctcggctgcaatcagaatcattgaaagcattagatggagctatagcttttgagcacaataatcctgatttgatctttgagttaggtgttcaatacgctgagcaacggaacttaaaagctgcttcccgttacgccaaagagttcatcgatgcaacgggagggtcagtgttaaaaggatggagatttctcgcgcttgttttgtcagctcaacaacggttttcagaagcagaagttgtgactgatgctgctttagatgaaactgcaaagtgggatcagggacctctcttgagactcaaagcaaagctgaaaatctctcagtcaaatccaacagaagccgttgagacttatcgttaccttcttgcattggttcaagcgcaaaggaaatctttcggacctctcagaactctttctcaggtttcaaaatctcttttgttctctaatctattgagatcatcaatcaaaacctttttttttttttattgaagaacttgtacaataacacagatggaggaagacaaagtgaatgagtttgaagtgtggcatggcttggcttatctttactcaagcctttcgcattggaacgacgtagaagtctgtctgaaaaaagccggagagctgaaacaatactctgcttcaatgttgcatacagaaggcaagaacaagaagccatttaactaatctcttagaatcatccattacttgatttactcaaatttggtttgaatctttgggaatgatcaggtcgaatgtgggaaggacgaaaggagttcaaacccgcgctagcagctttcttggacggtttattactagacggatcatcggttccttgcaaagtagcggttggagcgttattgtccgaaagagggaaagatcatcagccaactctccccgtggctagaagtttgctctctgatgcattgaggatcgatccaacaaaccgaaaagcttggtattacttaggaatggttcataaatctgatggacgtatagctgatgctactgattgcttccaagctgcttctatgcttgaagagtctgatcctattgaaagcttctcaaccattctttaa

SEQ ID NO: 9 - DNA, 2516 bp, Arabidopsis thaliana
  5′UTR (1)..(300)
  CDS (301)..(2412)
cgttttgcttctttttttttaaaacacattcttattataaatataaaaaaaacaaagagagatcaaaaacaaaaagtttcctctctttttctaattttttaaagtttctttcatcttcttcagatccgaattgtcgccgcgaaattcgtcagtgcagcttcttcttcttcgcgtactttattcgatcggctgtctgaagaacatgaagccgatgatcgtagttgctgctagttacggtggattgatgtttggatggacgcagaaattttgatgtgggtttagtctaaaaggtgaagaacaatgctcgggaatcaatccgcggattttagtgagaagggggaagatgagatcgtcagacagctttgtgctaatgggatttgcatgaaaacaactgaagttgaagcaaagcttgatgaaggaaatattcaagaagctgaatcttctttgagagaaggattatctctcaatttcgaggaagcaagagcacttcttggaagattggaataccaaagagggaatttagaaggcgcacttcgtgtctttgaaggtatcgaccttcaagcagctatccagcggttacaggtttccgtgcctcttgagaaaccggctactaagaaaaaccgtccccgtgaaccgcagcaatcagtttctcagcatgctgctaacttggtccttgaagctatctacttgaaagccaaatcccttcaaaagcttgggagaataactgaggctgctcatgaatgcaagagtgttcttgattctgttgagaagatatttcagcaagggataccagatgctcaagtggataacaaacttcaagaaaccgttagccacgccgttgaactacttcctgcgctatggaaagaatctggtgattatcaagaagccatatctgcttatagacgcgcgcttttaagccaatggaatcttgataatgattgttgtgcaaggattcaaaaagattttgcagtctttcttttacattctggagtcgaagcgagtccaccgagtttaggttctcagatagagggatcgtacatacctagaaacaacatagaagaagccattcttcttctaatgattcttttaaagaagtttaacctcgggaaagcgaaatgggatccgtctgtgtttgagcaccttacctttgcgttatctttatgtagtcagaccgcggttctcgccaagcagcttgaagaagtaatgcctggtgtgtttagccgtattgagcgttggaacactttggctctttcttatagtgcagcaggtcaaaacagtgctgcagttaaccttcttagaaagtctctgcataaacacgaacaacccgatgatcttgtggcgcttttgttagctgctaagctttgcagtgaagagccttctttagctgctgaaggtacgggttatgcgcagagagcgataaacaatgctcaaggtatggatgagcatttgaaaggcgttggtttgaggatgttaggactttgtttagggaaacaagcgaaggttccgacatcggattttgaaagatctcggctgcaatcagaatcattgaaagcattagatggagctatagcttttgagcacaataatcctgatttgatctttgagttaggtgttcaatacgctgagcaacggaacttaaaagctgcttcccgttacgccaaagagttcatcgatgcaacgggagggtcagtgttaaaaggatggagatttctcgcgcttgttttgtcagctcaacaacggttttcagaagcagaagttgtgactgatgctgctttagatgaaactgcaaagtgggatcagggacctctcttgagactcaaagcaaagctgaaaatctctcagtcaaatccaacagaagccgttgagacttatcgttaccttcttgcattggttcaagcgcaaaggaaatctttcggacctctcagaactctttctcagatggaggaagacaaagtgaatgagtttgaagtgtggcatggcttggcttatctttactcaagcctttcgcattggaacgacgtagaagtctgtctgaaaaaagccggagagctgaaacaatactctgcttcaatgttgcatacagaaggtcgaatgtgggaaggacgaaaggagttcaaacccgcgctagcagctttcttggacggtttattactagacggatcatcggttccttgcaaagtagcggttggagcgttattgtccgaaagagggaaagatcatcagccaactctccccgtggctagaagtttgctctctgatgcattgaggatcgatccaacaaaccgaaaagcttggtattacttaggaatggttcataaatctgatggacgtatagctgatgctactgattgcttccaagctgcttctatgcttgaagagtctgatcctattgaaagcttctcaaccattctttaaaaatccatcataaagatttatattcatatgttcattttttgtctgtcatctcatgtacaattttaaaccttcttaatttcttcaaactaaatatatattac

SEQ ID NO: 10 - protein, 704 aa, Arabidopsis thaliana
MLGNQSADFSEKGEDEIVRQLCANGICMKTTEVEAKLDEGNIQEAESSLREGLSLNFEEARALLGRLEYQRGNLEGALRVFEGIDLQAAIQRLQVSVPLEKPATKKNRPREPQQSVSQHAANLVLEAIYLKAKSLQKLGRITEAAHECKSVLDSVEKIFQQGIPDAQVDNKLQETVSHAVELLPALWKESGDYQEAISAYRRALLSQWNLDNDCCARIQKDFAVFLLHSGVEASPPSLGSQIEGSYIPRNNIEEAILLLMILLKKFNLGKAKWDPSVFEHLTFALSLCSQTAVLAKQLEEVMPGVFSRIERWNTLALSYSAAGQNSAAVNLLRKSLHKHEQPDDLVALLLAAKLCSEEPSLAAEGTGYAQRAINNAQGMDEHLKGVGLRMLGLCLGKQAKVPTSDFERSRLQSESLKALDGAIAFEHNNPDLIFELGVQYAEQRNLKAASRYAKEFIDATGGSVLKGWRFLALVLSAQQRFSEAEVVTDAALDETAKWDQGPLLRLKAKLKISQSNPTEAVETYRYLLALVQAQRKSFGPLRTLSQMEEDKVNEFEVWHGLAYLYSSLSHWNDVEVCLKKAGELKQYSASMLHTEGRMWEGRKEFKPALAAFLDGLLLDGSSVPCKVAVGALLSERGKDHQPTLPVARSLLSDALRIDPTNRKAWYYLGMVHKSDGRIADATDCFQAASMLEESDPIESFSTIL

SEQ ID NO: 11 - DNA, 31 bp, Artificial Sequence
  Description of Artificial Sequence Oligonucleotide primer
catgccatggtatgagtcgagtgtctgactt

SEQ ID NO: 12 - DNA, 28 bp, Artificial Sequence
  Description of Artificial Sequence Oligonucleotide primer
tcgccatggttcttcaccttttagacta

SEQ ID NO: 13 - DNA, 22 bp, Artificial Sequence
  Description of Artificial Sequence Oligonucleotide primer
atgctcgggaatcaatccgcgg

SEQ ID NO: 14 - DNA, 24 bp, Artificial Sequence
  Description of Artificial Sequence Oligonucleotide primer
ttaaagaatggttgagaagctttc

SEQ ID NO: 15 - DNA, 29 bp, Artificial Sequence
  Description of Artificial Sequence Oligonucleotide primer
accaaagagggaatttagaaggcgcactt

SEQ ID NO: 16 - DNA, 29 bp, Artificial Sequence
  Description of Artificial Sequence Oligonucleotide primer
tacgtaactcctagctaggttgtttggct

SEQ ID NO: 17 - DNA, 29 bp, Artificial Sequence
  Description of Artificial Sequence Oligonucleotide primer
cattttataataacgctgcggacatctac

SEQ ID NO: 18 - DNA, 29 bp, Artificial Sequence
  Description of Artificial Sequence Oligonucleotide primer
tgggaaaacctggcgttacccaacttaat

(19) United States
(12) Patent Application Publication
(10) Pub. No.: US 2003/0061635 A1
(43) Pub. Date: Mar. 27, 2003

(54) Pollen-specific novel calmodulin-binding protein, NPG1 (No Pollen Germination1), promoter, coding sequences and methods for using the same

(76) Inventors: Anireddy S.N. Reddy, Fort Collins, CO (US); Farida Safadi, Fort Collins, CO (US); Vaka S. Reddy, Fort Collins, CO (US); Maxim Golovkin, Philadelphia, PA (US)

Correspondence Address: GREENLEE WINNER AND SULLIVAN P C, 5370 MANHATTAN CIRCLE, SUITE 201, BOULDER, CO, 80303, US

(21) Appl. No.: 10/178,043

(22) Filed: Jun. 20, 2002

Related U.S. Application Data
(60) Provisional application No. 60/299,620, filed on Jun. 20, 2001.

(51) Int. Cl. A01H 1/00; C07H 21/04; C12N 9/50; C12P 21/02; C12N 5/04

(52) U.S. Cl. 800/287; 435/219; 435/69.1; 435/419; 435/320.1; 536/23.2

(57) ABSTRACT

The present disclosure describes a novel calmodulin-binding protein, termed NPG1 (No Pollen Germination1) of plants that is specifically expressed in pollen, and the nucleic acid coding sequence, and genomic DNA fragments containing pollen-specific transcriptional and translational regulatory elements. Genetic, histological, and pollen germination studies with the Arabidopsis mutant line for the NPG1 gene indicate that NPG1 is essential for pollen germination. Therefore, this invention provides new means to generate a transgenic plant that is male sterile by modulating expression of the NPG1 gene or interfering with the function of the NPG1 polypeptide. The availability of the tissue specific regulatory elements of the NPG1 gene makes it possible for the pollen-specific expression of various genes of interest.

### CROSS REFERENCE TO RELATED APPLICATIONS

This application claims priority from U.S. Provisional Patent Application Serial No. 60/299,620 filed Jun. 20, 2001.

### ACKNOWLEDGEMENT OF FEDERAL RESEARCH SUPPORT

The United States government has rights in this invention arising from Grant No. MCB-079938 from the National Science Foundation, Grant No. 970310 from the U.S. Department of Agriculture and Grant No. NAG10-0234 from the National Aeronautics and Space Administration that partially funded the research leading to this invention.

### BACKGROUND OF THE INVENTION

The present invention relates generally to the field of agricultural biotechnology. More particularly, the present invention concerns compositions and methods relating to a pollen specific calmodulin binding protein termed, NPG1 (No Pollen Germination1), nucleic acids encoding the NPG1 and a pollen specific promoter, and methods for using the same.

Calcium is an essential constituent of in vitro pollen germination media and a potential chemotropic agent guiding pollen tube growth in the transmitting tissue of the pistil (Brewbaker et al. (1963) Am. J. Bot. 50:859-865; Mascarenhas et al. (1964) Plant Physiol. 39:70-77; Badnarska, E. (1989) Sex. Plant Reprod. 2:53-58; Badnarska et al. (1995) Folia Histochem. Cytobiol. 33:43-52; Picton et al. (1983) Protoplasma. 115:11-17; and Steer et al. (1989) New Phytol. 111:323-358). High Ca²⁺ concentrations have been reported in the vicinity of the germination apertures of hydrated pollen (Tirlapur et al. (1992) Ann. Bot. 69:503-508). Studies with ⁴⁵Ca²⁺ (Badnarska, E. (1989) supra; Jaffe et al. (1975) J. Cell. Biol. 67:488-492) and later direct measurement of cytosolic Ca²⁺ concentration ([Ca²⁺]_{cyt}) using fluorescence ratio imaging of Ca²⁺ indicator dyes established that, in pollen tubes, [Ca²⁺]_{cyt} accumulates at the growing tip and forms a steep-tip focused gradient with about 3.0 μM at the tip to about 0.2 μM within 20 or 65 μm from the tip (Steer et al. (1989) supra, Rathore et al. (1991) Dev. Biol. 148:612-619; Miller et al. (1992) J. Cell. Sci. 101:7-12; Malho et al. (1994) Plant J. 5:331-341; Pierson et al (1994) Plant Cell 6:1815-1828; Bibikova et al. (1997) Planta 203:495-505; Herth et al. (1990) Tip Growth in Plant and Fungal Cells (Heath I. B., ed) pp. 91-118, Academic Press, New York; Pierson et al. (1992) Int. Rev. Cytol. 140:73-125; and Franklin-Tong, V. E. (1999) Plant Cell 11:727-125). Pollen tube elongation is inhibited by chemicals (e.g. ion channel blockers or ionophores) that interfere with Ca²⁺ homeostatis in the growing pollen tubes (Badnarska, E. (1989) supra; Malho et al. (1994) supra; Pierson et al. (1994) supra; Herth et al. (1990) supra; Obermeyer et al. (1991) Eur. J. Cell Biol. 56:319-327; Picton et al. (1985) Planta (Basel) 163:20-26; and Heslop-Harrison et al. (1992) Ann. Bot. 69:395-403). Disrupting the Ca²⁺ gradient or blocking Ca²⁺ influx inhibited pollen tube growth, dissipated the cytoplasmic streaming associated with the pollen tube elongation and eliminated the clear zone found at the tip of actively elongating pollen (Tirlapur et al. (1992) supra, Pierson et al. (1994) supra; and Pierson et al. (1990) J. Exp. Bot 41:1461-1468). Extracellular Ca²⁺ influx at the pollen tube tip was reported and this influx was correlated with the intracellular Ca²⁺ gradient and polarized growth of the tip (Pierson et al. (1994) supra; Reiss et al. (1985) Planta 163:84-90; Malho et al. (1995) Plant Cell 7:1173-1184; and Kuhtreiber et a. (1990) J. Cell Biol. 110:1565-1573). Ca²⁺ influx at the apex is speculated to be due to stretch-activated Ca²⁺ channels at the tip (Pierson et al. (1994) supra; and Malho et al. (1995) supra) while Ca²⁺-ATPase pumps are implicated in maintaining the Ca²⁺ gradients in the cytosol (Tirlapur et al. (1992) supra; and Obermeyer et al. (1991) supra). Recently, the relationship between [Ca²⁺]_{cyt} and pollen tube growth was further strengthened by the discovery that the tip-focused (Ca²⁺]_{cyt} gradients oscillate with high [Ca²⁺]_{cyt} corresponding to peaks in pollen tube growth (Holdaway-Clarke et al. (1997) Plant Cell 9:1999-2010; Calder et al. (1997) Biochem. Cell Biol. 234:690-694; Messerli et al. (1999) J. Cell. Sci. 112:1497-1509; and Pierson et al. (1996) Dev. Biol. 174:160-173). Furthermore, Ca²⁺ has been shown to influence the direction of pollen tube growth. Induced Ca²⁺ fluctuation in the cytosol or modifying extracellular Ca²⁺ influx redirected pollen tube growth toward the high Ca²⁺ concentration (Bibikova et al. (1997) supra; Pierson et al. (1996) supra; and Malho et al. (1996) Plant Cell 8:1935-1949), suggesting a role for pistil-derived Ca²⁺ in the directed growth of pollen tubes toward the ovary. These studies showed that the establishment and maintenance of a precise tip-focused intracellular Ca²⁺ gradient, possibly through regulating Ca²⁺ influxes at the tip, is essential for pollen tube elongation and directional growth. How the tip-focused Ca²⁺ gradient at the tip regulates pollen tube growth and direction is poorly understood. Some studies suggest that Ca²⁺ interacts directly or indirectly, through Ca²⁺-binding proteins, with the cytoskeleton to regulate cytoplasmic streaming, vesicle fusion, and the function of cytoskeletal elements required for tube emergence and growth (Steer et al. (1989) supra; Pierson et al. (1994) supra; Picton et al. (1985) supra; Heslop-Harrison et al. (1992) supra; Malho et al. (1996) supra; Heslop-Harrison et al. (1989) J. Cell. Sci. 94:391-325; Ma et al. (1999) Plant Cell 11:1351-1363; Derksen et al. (1995) Acta Bot. Neerl. 44:93-119; Mascarenhas, J. P. (1993) Plant Cell 5:1303-1314; Cai et al. (1997) Trends Plant Sci. 2:86-91; Taylor et al. (1997) Annu. Rev. Plant Physiol. Plant Mol. Biol. 48:461-491; and Heslop-Harrison, J. (1987) Int. Rev. Cytol. 107:1-78).

Calcium has been implicated in regulating diverse physiological processes in plants (Trewavas, et al. (1991) Trends Genet. 107:356-361; Trewavas et al. (1998) Curr. Opin. Plant Biol. 1:428-433; Sanders, et al. (1999) Plant Cell 11:691-706; Poovaiah et al. (1993) CRC Crit. Rev. Plant Sci 12:185-211; and Bush, D. S. (1995) Annu. Rev. Plant Physiol. Plant Mol. Biol. 46:95-122). Calcium-regulated physiological responses are often mediated directly or indirectly by Ca²⁺-modulated proteins of which Calmodulin (CaM) is ubiquitous in all eukaryotes. Calmodulin, a Ca²⁺- modulated protein with four Ca²⁺-binding EF-hands, is considered to be the primary intracellular Ca²⁺ receptor in all eukaryotes. In plants, over the last 10 years CaM and CaM isoforms have been identified, and their involvement in transducing Ca²⁺ signals into a variety of cellular responses has been reported (Roberts et al. (1992) Annu. Rev. Plant Physiol. Plant Mol. Biol. 43:375-414; Snedded et al. (1998) Trends Plant Sci. 3:299-304; and Zielinski, R. E. (1998) Annu. Rev. Plant Physiol. Plant Mol. Biol. 49:95-122). Since CaM acts by modulating the activity of a variety of other proteins directly by interacting with them, research in recent years has focused on identifying the CaM-target proteins and analyzing the function of these proteins in cellular processes. Whereas a wide variety of CaM-activated proteins were described and characterized in animals, only a few CaM-binding proteins have been identified in plants and their function in regulating plant growth and development in response to elevated Ca²⁺ signals is still in its infancy (Snedden et al. (1998) supra; Zielinski, R. E. (1998) supra; and Reddy and Reddy (2002) in Handbook of Plant and Crop Physiology (Pessarakli, M., ed.) Marcel Dekker Inc., New York, pages 697-732).

Considering the widespread association between CaM and Ca²⁺-sensitive cellular processes, it is reasonable to expect CaM to mediate Ca²⁺ action in pollen tubes. Some reports suggest the involvement of CaM in mediating Ca²⁺ effect on pollen tube growth (Trewavas et al. (1998) Curr. Opin. Plant Biol. 1:428-433). Exogenous CaM enhances pollen germination and pollen tube growth (Ma et al. (1999) supra; Polito, V. S. (1983) in Pollen: Biology and Implications for Plant Breeding (Mulcahy, D. L., and Ottoviano, E., eds.) pp. 53-60, Elsevier Science Publishing Co., Inc., Amsterdam), whereas CaM antagonists and anti-CaM serum inhibit pollen germination and tube growth (Picton et al. (1985) supra; Polito, V. S. (1983) supra; Ma et al. (1997) Planta (Basal) 202:336-340) and stop cytoplasmic streaming (Obermeyer et al. (1991) Eur. J. Cell Biol. 56:319-327) in a concentration-dependent manner. In addition, upon CaM antagonist treatment of pollen tubes, Ca²⁺ remains in the tip membranes (Polito, V. S. (1983) supra) or its level increases behind the tip (Obermeyer et al. (1991) supra), suggesting that CaM is involved in maintaining Ca²⁺ gradients in the pollen tubes through Ca²⁺ influx from the plasma membrane channels and/or sequestration of Ca²⁺ into the internal organelles by CaM-regulated Ca²⁺-ATPases (Taylor et al. (1997) supra; and Hepler, P. K. (1997) Trends Plant Sci. 2:79-80). Ca²⁺-ATPase activity has been detected in the plasma membrane of the pollen tube tip as well as in the endoplasmic reticulum and mitochondria behind the tip and these ATPases are stimulated by CaM (Tirlapur et al. (1992) supra; Zielinski, R. E. (1998) supra; and Rasi-Caldogno et al. (1989) Plant Physiol. 90:1429-1434). Calmodulin localization in the pollen during various stages of pollen growth has produced contradicting results. Calmodulin is localized to the region of germinal apertures of the hydrated pollen, the plasma membrane, and the cytoplasm in the vicinity of the germination bubble and in the plasma membrane and the cytosol of the growing pollen tube where it forms an apically focused gradient similar to the tip-focused Ca²⁺ gradient (Tirlapur et al. (1992) supra; and Tirlapur et al. (1994) Zygote 2:63-68). A similar localization of CaM is observed in the Fucus rhizoid tip pre-emergence location (Trewavas et al. (1998) supra), suggesting a role for CaM in polar growth and tip extension. In other localization studies, however, CaM was found to be diffuse and uniformly distributed in the pollen tube (Hausser et al. (1984) Planta (Basel) 162:33-39; and Reddy et al. (1996) J. Bio. Chem. 11:131-139) and no tip high gradient of the protein was observed (Reddy et al. (1996) supra). Recent studies on effects of exogenous CaM on pollen tube germination and growth suggested that CaM acts extracellularly in exerting its effect on pollen possibly through a signal transduction pathway involving a receptor-mediated stimulation of G protein (Ma et al. (1999) supra; and Ma et al. (1997) supra).

Although these studies implicate the involvement of CaM in pollen germination and tube growth, little is known about proteins that bind to CaM in pollen. Hence studies on pollen-specific CaM-binding proteins should help us understand the role of CaM in Ca²⁺-mediated signal transduction pathways in pollen. Furthermore, identification of a pollen-specific protein that mediates Ca²⁺ dependent pollen tube growth could allow the regulation of male fertility of plants because the pollen-specific protein is required for pollen germination. Male-sterile plants are of use in agricultural biotechnology for control of plant breeding, particularly for plant species such as maize where commercially grown hybrid seed is generated by controlled crossing of different inbred lines. Production of male-sterile plants would allow the plant breeder to have greater control over the fertilization process, for example to prevent self-fertilization of a plant. At present, such control for corn plants exerted by detasseling the plants to be fertilized, an expensive and time-consuming process. Therefore, there is need in the field for a reliable method of inducing male sterility in plant species. A pollen specific promoter would also be of use in agricultural biotechnology. It may be desirable to express a particular gene only in the pollen of transgenic plants, for example, a transgenic conferring male sterility or reduced allergen production. Because regulatory proteins may have unknown effects in various plant tissues, the tissue specific expression of transgenes would be of substantial use. A need exists in the field for tissue specific plant promoters, such as a pollen specific promoter.

### SUMMARY OF THE INVENTION

The present invention provides a novel pollen-specific CaM-binding protein termed NPG1 (No Pollen Germination1), cDNA1 and genomic DNA sequences encoding the NPG1 and an associated pollen-specific promoter. The NPG1 polypeptide is shown to be expressed specifically in pollen and essential for pollen germination. Therefore, the availability of the NPG1 polypeptide, the coding sequence, and the genomic fragment containing the transcription and translation regulatory elements offers new means of regulating male fertility in plants.

Specifically exemplified herein are the NPG1s from maize and Arabidopsis. However, based on the high degree of amino acid sequence homology (54% identity and 68% similarity) between the maize and Arabidopsis NPG1 polypeptides, it is expected that the NPG1 gene is highly conserved among plants. Therefore, a person of ordinary skill in the art can isolate a NPG1 homolog from other plants using the nucleotide and amino acid sequences disclosed in the present application.

The NPG1 polypeptides from maize and Arabidopsis contain multiple structural motifs known as tetratricopeptide repeats (TPRs), three in the maize NPG1 and six in the Arabidopsis version. The TPR motifs are known to function in protein-protein interaction. Thus, it is possible that the NPG1 peptide interacts with other cellular proteins that are yet to be identified. A skilled artisan can use the NPG1 polypeptide of maize or Arabidopsis (or fragments thereof) to isolate a protein(s) that interact with the NPG1 polypeptide according to the methods described herein.

The NPG1 polypeptide binds calmodulin (CaM) in a calcium-dependent manner. The region of the NPG1 polypeptide that binds to CaM is mapped to an 18 amino acid stretch as shown in FIG. 1. The identification of the CaM binding domain in the NPG1 offers a means to identify compositions that can activate or inhibit the interaction between CaM and the NPG1 protein. For example, this interaction can be inhibited by a peptide derived from the NPG1 protein (e.g., 18-mer identified herein), anti-NPG1 antibody that specifically recognizes the 18 amino acid stretch or a small molecule.

The nucleic acid encoding NPG1 can be used to produce recombinant NPG1 polypeptide or fragments thereof by employing the recombinant DNA technology readily available in the art. The expressed proteins or peptides can be used as antigens to raise antibodies specific for NPG1 or as a ligand to identify and/or isolate additional proteins that interact with the NPG1 peptide. The peptides derived from the NPG1 polypeptide (e.g., the 18 amino acid fragment that binds to CaM) can also be made using the art-known peptide synthetic methods.

The present invention also provides pollen-specific transcriptional regulatory elements that are useful for expressing a gene of interest in a pollen-specific manner. For example, an antisense nucleotide of at least 10 nucleotides in length complementary to the NPG1 mRNA or a fragment thereof can be expressed specifically in pollen to reduce the level of the NPG1 polypeptide. Since the NPG1 gene is required for pollen germination, disruption of the NPG1 gene function will result in male sterile plants. A similar antisense strategy can be applied to reduce allergen production in pollen.

The fact that the NPG1 polypeptide is essential for pollen germination offers new possibilities of generating male-sterile plants by numerous art-known methods. These include, but are not limited to, introducing a mutation in the NPG1 coding sequence, down-regulating the expression of the NPG1 gene, or inactivating the polypeptide or introducing an inhibitory composition that interrupts NPG1 protein function (e.g., NPG1-derived peptides or NPG1-specific antibodies).

### BRIEF DESCRIPTION OF THE DRAWINGS

FIGS. 1A-1C illustrate the structure of the maize NPG1 gene. FIG. 1A shows the genomic DNA sequence of the NPG1 gene from maize along with the encoded amino acid sequence of the NPG1 polypeptide. Exons are indicated in uppercase letters and introns, 5′ and 3′ non-coding regions in lowercase letters. The entire genomic DNA sequence is provided as SEQ ID NO:3, while the encoded amino acid sequence of NPG1 is provided as SEQ ID NO:1. FIG. 1B shows the exonic structure of the NPG1 gene from maize, indicated by the numbered boxes. FIG. 1C shows the relative location of various structural features of maize NPG1 polypeptide. TPR sequences were identified using the SMART program (Schultz et al. (1998) Proc. Natl. Acad. Sci. U.S.A. 95:5857-5864). The CaM-binding domain was identified by testing the binding of truncated proteins of NPG1 and a synthetic peptide to CaM. The sequence of the CaM binding domain is provided as SEQ ID NO:2.

FIG. 2 shows an alignment of the amino acid sequence of maize NPG1 polypeptide and comparison with two proteins termed AtNPG1 and 2 in the Arabidopsis genome database (accession numbers At2g43040 and At1g27460). Identical amino acids are shown by reverse lettering. Dashes indicate gaps in the alignment. The three TPRs and the CaM-binding domains are indicated by a single and a double line over their corresponding residues, respectively.

FIG. 3 shows the mapping of the CaM-binding domain in maize NPG1. The schematic diagram of the partial cDNAs of NPG1 that were expressed in bacteria using pET28 to map the CaM-binding domain. Filled bar indicates the region of cDNA containing the CaM-binding domain. Construct P3 represents the cDNA isolated with labeled CaM. Constructs 2, 3, 4, 5, and 6 are truncated versions of P3. The expression of the different fusion proteins was induced by IPTG and the fusion proteins analyzed for their binding to T7 tag monoclonal antibody, biotinylated CaM (Bio-CaM), and ³⁵S-CaM. The results of the binding assays are shown on the right side of FIG. 3.

FIGS. 4A-4B demonstrate that CaM binds to maize NPG1. FIG. 4A is the analysis of CaM binding to a synthetic 18-amino acid peptide (MP-1, SEQ ID NO:2) by electrophoretic mobility shift polyacrylamide gels with 4 M urea. Lane 1 contained CaM alone (166 pmol), lanes 2-4 contained CaM (166 pmol) and 166, 332 and 664 pmol, respectively of synthetic MP-1. BCaM is bovine CaM, CaM2, CaM4, and CaM6 are the 2, 4, and 6 isoforms of Arabidopsis CaM. FIG. 4B shows the fluorescence emission profiles of synthetic MP-1 in the presence or absence of BCaM, CaM2, CaM4, or CaM6. CaM and MP-1 (166 pmol) were incubated in 5 mM Tris, pH 7.3 and 0.5 mM CaCl₂. The excitation was measured at 290 nm and bandwidth for excitation and emission was 5 nm.

FIG. 5 shows the nucleotide and deduced amino acid sequence of Arabidopsis NPG1. Exon and introns are presented in upper and lowercase letters, respectively. The deduced amino acid sequence is shown under the nucleotide sequence. Numbers at right correspond to nucleotides and amino acids (bold). The CaM-binding domain is indicated by reverse lettering. Gray boxes indicate six putative tetratricopeptide repeats.

FIG. 6 illustrates that Arabidopsis NPG1 is a CaM-binding protein. Binding of bacterially expressed and purified NPG1 (Coomassie) to horseradish peroxidase labeled Arabidopsis CaM isoform-2 (CaM-2), -4 (CaM-4), and -6 (CaM-6) in the absence (−Ca²⁺) or presence of calcium (+Ca²⁺).

FIGS. 7A-7D demonstrate that NPG1 is expressed only in Arabidopsis pollen. FIG. 7A shows Northern blot analysis. The blot was first hybridized with NPG1 cDNA (top) and then with the Arabidopsis ubiquitin (Ubq) gene (bottom). FIG. 7B shows the results of amplification of NPG1 transcript in pollen and tissues by RT-PCR (top). Amplified product using the same primers with genomic DNA (DNA) is also shown. The presence of cDNA template in all reactions was confirmed by amplification of the cyclophilin gene (bottom). FIG. 7C shows the expression of GFP fused to the NPG1 promoter. GFP expression in transiently transformed germinating tobacco pollen. FIG. 7D shows the expression of GFP fused to the NPG1 promoter in mature (top), or germinated pollen (bottom) from a heterozygous transgenic Arabidopsis plant.

FIGS. 8A-8E illustrate the results of the Arabidopsis NPG1 mutant characterization and complementation studies. FIG. 8A is a schematic diagram of NPG1 gene showing the site of T-DNA insertion. Numbered boxes represent exons. Coding region upstream of T-DNA is shown in dark gray. The position of the CaM-binding domain (CBD), as defined by deletion analysis, is indicated as a dark oval underneath exon 3. Tetratricopeptide repeat motifs are shown in light gray boxes. FIG. 8B shows the results of polymerase chain reaction with the DNA from a NPG1/npg1 plant using gene-specific forward and reverse primers (NPG), and with NPG1 reverse primer and T-DNA left border (T-DNA). FIG. 8C shows the Southern blot analysis with DNA from wild type and NPG1 mutant. Genomic DNA of wild type (WT) and NPG1 mutant digested with BamHI (single site in T-DNA and no site in NPG) was hybridized first with the full-length NPG1 cDNA (NPG probe) and then with T-DNA that contains a BamHI site (T-DNA probe). FIG. 8D shows the analysis of progeny from a cross between male sterile mutant (cer6-2) and NPG1 mutant for kanamycin resistant seedlings. Kan−, plates without kanamycin, Kan+, plates with 50 μg/ml kanamycin. FIG. 8E illustrates the complementation studies of NPG1 mutation. Pollen from NPG1 mutant plants additionally containing a NPG1 cDNA driven by the NPG1 promoter was used to pollinate flowers of a male sterile mutant cer6-2. Seeds from this cross were grown on a medium containing kanamycin and basta (Kan⁺, BASTA⁺).

FIGS. 9A-9C show pollen development in NPG1 mutant. FIG. 9A shows the sections of Arabidopsis flower (stages 7 to 8 according to Sanders et al.(1999) Sex Plant Reprod. 11:297-322) from NPG1 mutant (top). Magnified view of the anther (middle) and tetrad (bottom). FIG. 9B shows pollen development through stages 9 (top), 10 (middle), and 11 (bottom). FIG. 9C shows DAPI staining of pollen from NPG1 mutant. White arrowheads indicate the diffusely staining vegetative nucleus and densely staining two sperm cell nuclei (top). All DAPI stained pollen grains from the mutant (NPG1/npg1) have one vegetative nucleus and two sperm nuclei (bottom).

FIGS. 10A-10C show pollen development and germination in double mutant (NPG/npg, qrt/qrt). FIG. 10A is DAPI staining of a tetrad from NPG1 mutant (NPG1/npg1) in the qrt background. White arrows point to the vegetative nucleus and two sperm nuclei in each pollen grain of a tetrad. FIG. 10B is the SEM image of a tetrad from double mutant (NPG1/npg1, qrt/qrt). FIG. 10C shows the germination of tetrads from qrt/qrt and NPG1/npg1, qrt/qrt plants. The number of germinated pollen from each tetrad was counted. Tetrads with 1 or 2 germinating pollen (1+2) and 3 or 4 germinating pollen (3+4) from the quartet mutant (qrt/qrt) and the npg1 mutant in quartet background (NPG1/npg1, qrt/qrt ) are shown (top). The maximum number of germinating pollen in a tetrad from qrt/qrt is four (bottom left) while the maximum number for an NPG1/npg1, qrt/qrt mutant is two (bottom right).

### DETAILED DESCRIPTION OF THE INVENTION

In general, the terms and phrases used herein have their art-recognized meaning, which can be found by reference to standard textbooks, journal references, and contexts known to those skilled in the art. The following definitions are provided to clarify their specific use in the context of the present invention.

The term “pollen-specific calmodulin binding protein” refers to a protein that is expressed specifically in pollen and binds calmodulin in a calcium dependent manner. This term is intended to include a homolog of the maize and Arabidopsis NPG1 polypeptide in other plants. “NPG1 (No Pollen Germination1)” disclosed herein is an example of the pollen-specific CaM binding protein in maize and Arabidopsis. It is termed as NPG1 because this protein is required for pollen germination and the plant containing a mutation, that makes the NPG1 protein nonfunctional, does not germinate.

“Tissue specific gene expression” as used herein means that a given gene is expressed differentially among the tissues in an organism and often expressed in certain tissues only. For example, the NPG1 is expressed specifically in pollen and not in other tissues of a plant. The expression of a gene in a tissue specific manner is controlled by the regulatory elements generally present at the 5′ flanking region of the gene. “Regulatory elements” refer to a DNA fragment containing nucleotide sequences generally known as the promoters, enhancers, introns, polyadenylation signal sequences, terminators, ribosome binding sequences and the like that are effective for expressing a gene of interest operably linked thereto.

“Expression vector” refers to a DNA construct containing a nucleic acid coding for a gene product of interest in which part or all of the nucleic acid coding sequence is capable of being transcribed and/or translated in a cell that harbors such construct. Typically, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. Expression vectors generally contain a coding sequence for a selectable marker gene (e.g., antibiotic resistance gene) such that a cell harboring the vector can be readily screened in the presence of a selectable composition.

The phrase, “operably linked to” is intended to mean that a coding sequence for a gene of interest is linked to a transcriptional and translational regulatory sequences in such a way that a gene is capable of being transcribed and translated properly.

A “host cell” refers to a cell that contains a exogenously introduced DNA construct to express a gene of interest.

“Epitope” refers to a region of a molecule that elicits an immune response and recognized by a given set of antibodies.

The term “mutation” as used herein is intended to indicate a variety of changes to render a gene of interest non-functional. These include point, deletion, and substitution mutations in the coding sequence of the gene or insertion of a foreign DNA within the gene as well as a similar type of mutation in the regulatory sequence that affects expression of the gene.

“Antisense strategy” refers to a technology that is used to interfere with the expression of a gene of interest by employing a nucleic acid molecule complementary to the sense sequence i.e., antisense, of the gene of interest. Typically, an antisense molecule is introduced into a cell or tissue, transiently or stably. If the antisense strategy is effective, i.e., the introduced nucleic acid molecule interferes with the expression of the gene, the level of the mRNA and/or protein encoded by the gene of interest is reduced to yield the phenotype desired.

A “homolog” of a maize or Arabidopsis NPG1 gene is a gene sequence encoding a NPG1 polypeptide isolated from an organism other than maize or Arabidopsis.

A cell, tissue, organ, or organism into which a foreign nucleic acid has been introduced, such as an expression vector, is considered to be “transformed”, “transfected”, or “transgenic”. A transgenic or transformed cell or organism also includes progeny of the cell or organism and a progeny produced from a breeding program employing such a transgenic plant as a parent in a cross and exhibiting an altered phenotype resulting from the presence of the recombinant DNA construct.

Using the nucleotide and the amino acid sequence of the NPG1 polypeptides disclosed herein, those skilled in the art can create DNA molecules and polypeptides that have minor variations in their nucleotide or amino acid sequence. “Variant” DNA molecules are DNA molecules containing minor changes in a native NPG1 sequence, i.e., changes in which one or more nucleotides of a native NPG1 sequence is deleted, added, and/or substituted, while substantially maintaining NPG1 biological function.

As used herein, percent sequence identity of two nucleic acids is determined using the algorithm of Karlin and Altschul (1990) Proc. Natl. Acad. Sci. USA 87:2264-2268, and modified as in Karlin and Altschul (1993) Proc. Natl. Acad. Sci. USA 90:5873-5877. Such an algorithm is incorporated into the NBLAST and XBLAST programs of Altschul et al. (1990) J. Mol. Biol. 215:402-410. BLAST nucleotide searches are performed with the NBLAST program, score=100, wordlength=12, to obtain nucleotide sequences with the desired percent sequence identity. To obtain gapped alignments for comparison purposes, Gapped BLAST is used as described in Altschul et al. (1997) Nucl. Acids. Res. 25:3389-3402. When utilizing BLAST and Gapped BLAST programs, the default parameters of the respective programs (NBLAST and XBLAST) are used. See, for example, the National Center for Biotechnology Information website on the internet.

Sexual reproduction in flowering plants depends on pollination and fertilization. After landing on the stigma, pollen germinates and delivers sperm cells to the embryo sac by growing through the transmitting tissue in the style. The mechanisms controlling these processes are not understood. Despite numerous studies on the role of calcium and calmodulin (CaM) in pollen germination and pollen tube growth, the proteins that mediate calcium and calmodulin action have not been identified. The present application discloses a novel calmodulin binding protein, termed NPG1 (No Pollination Germination1), in maize and Arabidopsis that is found to be specifically expressed in pollen. This protein is termed as NPG1 based on the genetic, histological, and pollen germination studies described herein that indicate that NPG1 is essential for pollen germination and that the pollen carrying the non-functional NPG1 is not transmitted through male gametophyte.

In order to identify a protein(s) that interact with calmodulin in maize pollen, a protein-protein interaction-based screening was used to isolate cDNAs encoding such proteins. A cDNA library from maize pollen prepared in X Zap II expression vector was screened with plant CaM isoforms described in Liao and Zielinski (1995) Methods Cell Biol. 49:487-500. Screening of about 120,000 recombinants resulted in the isolation of 6 clones coding for putative maize pollen NPG1. To confirm the Ca²⁺-dependent binding of CaM, the putative clones were probed with ³⁵S-CaM in the presence of Ca²⁺ or EGTA, a Ca²⁺ chelator. All clones showed binding to ³⁵S-CaM in the presence of Ca²⁺ but not in the presence of EGTA. Restriction enzyme analysis and the length of cDNAs revealed that they were all derived from the same clone. Sequencing of the cDNAs indicated that all six cDNAs were identical. The 1.2 kb cDNA included part of the complete maize NPG1 cDNA sequence, shown in SEQ ID NO:7. The amino acid sequence of the protein encoded by the NPG1 gene is shown in SEQ ID NO:1.

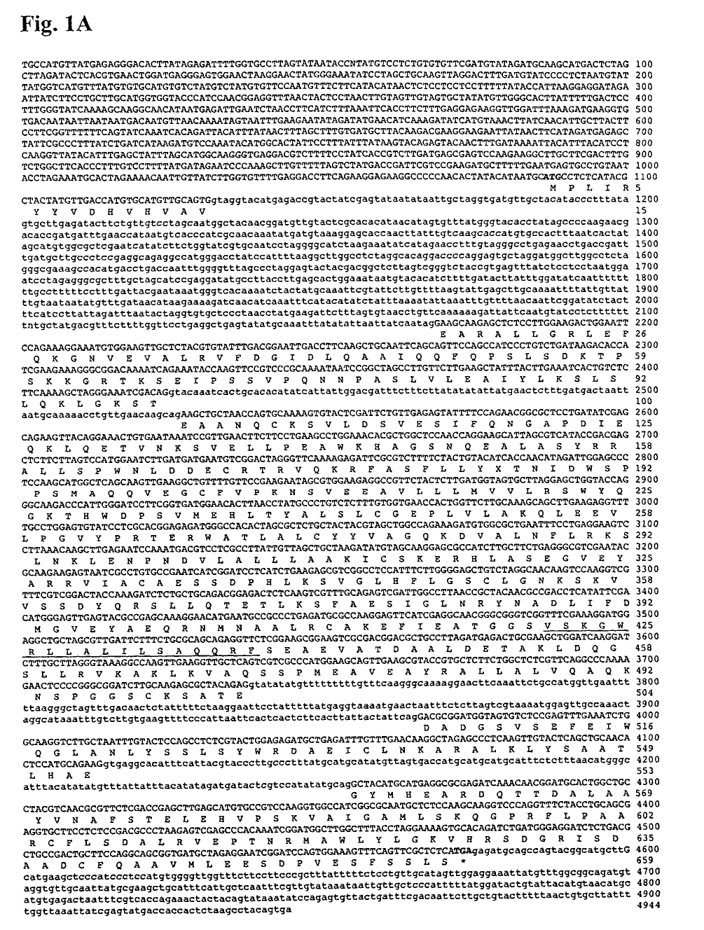

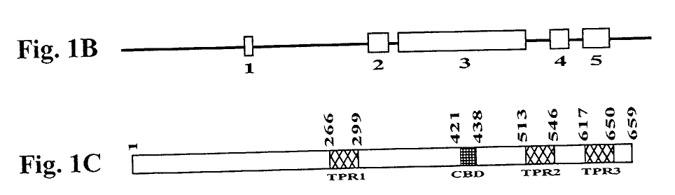

Since the isolated cDNA (1.2 kb) was much smaller than the transcript size estimated by Northern blot analysis, a genomic library was screened to isolate the full-length gene. Screening of maize genomic library with the partial cDNA clone (1.2 kb) yielded several positives. All positive clones were characterized by restriction mapping and Southern analysis. The clone containing the largest hybridizing band was sequenced by primer walking. Introns in the NPG1 gene were predicted by comparing the gene sequence with the cDNA and by using the NetPlantGene and NetGene2 programs (Hebsgaard et al. (1996) Nuc. Acid Res. 24(17):3439-3452; Brunak et al. (1991) J. Mol. Biol. 220:49-65). The nucleotide sequence of the maize NPG1 gene (SEQ ID NO:3) and the encoded amino acid sequence (SEQ ID NO:1) are shown in FIG. 1A. The maize NPG1 gene has 5 exons and 4 introns (FIGS. 1A and 1B). The open reading frame of NPG1 starts with a translation initiation codon in exon 1 (nucleotide position 1087 in FIG. 1A) and ends with a stop codon (TGA) in exon 5 (nucleotide position 4572 in FIG. 1A).

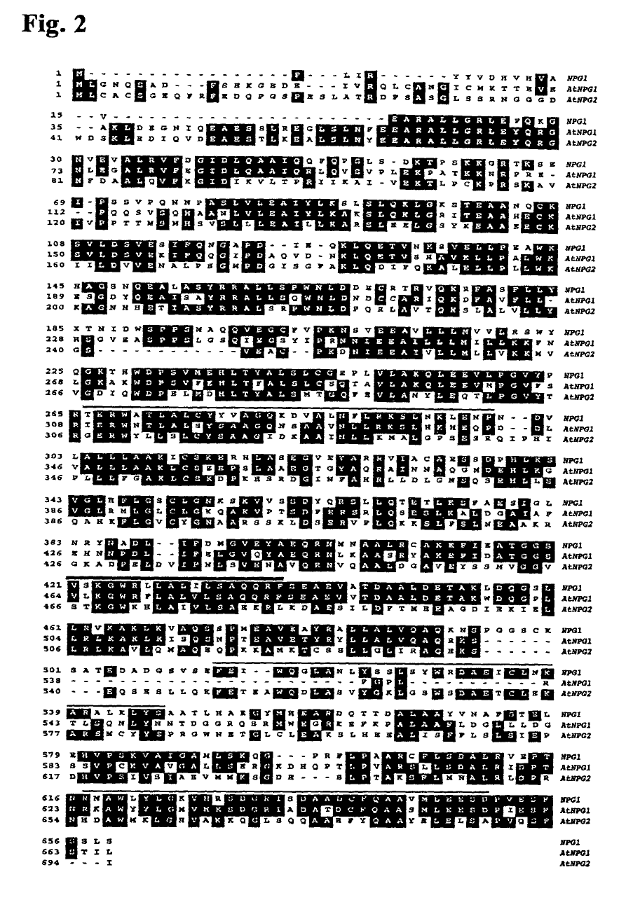

The size of the added exons or cDNA (1980 bp, SEQ ID NO:7) was in agreement with the estimated size of the transcript on Northern blot analysis. The encoded protein has 659 amino acid residues with an estimated molecular mass of about 72 kDa. A search of sequence databases with the encoded amino acid sequence (SEQ ID NO:1) using BLAST searches revealed that the protein was homologous to two hypothetical proteins found in the Arabidopsis genome database. A protein with accession number At2g43040 showed 54% identity and 68% similarity, and another protein (accession number At1g27460) showed 39% identity and 56% similarity. Both of those proteins have similar gene structure with five exons. The predicted CaM-binding domain in NPG1 was conserved in both of those proteins (FIG. 1C and FIG. 2). Based on this high degree of sequence identity, the Arabidopsis proteins were named AtNPG1 (accession number At2g43040) and AtNPG2 (accession number Atlg27460).

AtNPG1 and AtNPG2 are located on chromosome 2 and chromosome 1, respectively. In addition, maize NPG1 showed limited sequence similarity to SPINDLY, a protein involved in Giberellic acid (GA) signal transduction from Arabidopsis (U62135; 34% identity and 57% similarity) (Jacobsen et al. (1996) Proc. Natl. Aca. Sci. U.S.A. 93:9292-9296), and barley (AF035820; 19% identity and 35% similarity) (Robertson et al., 1998 Plant Cell 10:995-1007), and to an O-linked GlcNAc transferase from Methanobacterium thermoautotrophicum (Smith et al. (1998) Trends Plant Sci. 3:299-304) with 19% identity and 37% similarity.

FIG. 2 shows the alignment of the deduced amino acid sequence of maize NPG1 with the amino acid sequences of AtNPG1 and AtNPG2, two hypothetical proteins identified in the database. SPINDLY was not included in the alignment as it is a much larger protein (914 amino acids) and showed limited homology. Analysis of the encoded amino acid sequence of maize NPG1 using SMART (Simple Modular Architecture Research Tool), a program that predicts functional domains (Schultz et al. (1998) supra) revealed the presence of three TPRs, one in each of exons 3, 4, and 5 (FIG. 1C and FIG. 2). TPR (tetratricopeptide repeat) domains consist of degenerate consensus sequences and are implicated in protein-protein interaction (Lamb et al. (1995) Trends Biochem. Sci. 20:257-259).

TPRs from different proteins have loosely conserved consensus residues that are conserved in terms of their size, hydrophobicity, and spacing in a way to allow the motif to form a pair of antiparallel amphipathic α-helices (Lamb et al. (1995) supra; Sikorski et al. (1990) Cell 60:307-317; Das et al. (1998) EMBO J. 17:1192-1199). These helices are predicted to form the so-called “knob and hole” model which is responsible for protein-protein interaction either intramolecularly among the multiple repeats or intermolecularly by interacting with TPR or non-TPR target proteins (Lamb et al. (1995) supra; Sikorski et al. (1990) supra; Das et al. (1998) supra). One of the features of TPR sequences is that even though they are loosely conserved with a consensus sequence, there is variation among different TPRs, implying that TPR motifs interact with diverse proteins. In support of this, TPR proteins have been shown to interact with different target proteins (Das et al. (1998) supra; Prodromou et al. (1999) EMBO J. 18:754-762; Venolia et al. (1999) Cell Motil. Cytoskeleton 42:163-177). Furthermore, mutation or deletion of the TPRs has, in all cases, resulted in loss of function (Lamb et al. (1995) supra; Venolia et al (1999) supra).

The observation that TPRs are present in proteins with a wide array of functions and in various combinations and numbers has led to the assumption that TPR functions as a scaffold in binding to specific substrates depending upon the secondary structure assumed by the individual or combination of TPRs (Lamb et al. (1995) supra; Das et al. (1998) supra; Venolia et al. (1999) supra). The functions reported in the literature all require that the TPR-containing proteins form a complex with another component to regulate their function (Lamb et al. (1995) supra; Sikorski et al. (1990) supra; Kragler et al. (1998) Proc. Natl. Acad Sci. U.S.A. 95:13336-13341; Prodromou et al. (1999) supra; Venolia et al. (1999) supra; Vucich and Gasser (1996) Mol Gen. Genet. 252:510-517; and Hernandez Torres et al. (1995) Plant Mol. Biol. 27:1221-1226).

The proteins that are similar to maize NPG1 contain one or more TPRs. The alignment of TPR1, -2, and -3 of maize NPG1 with TPRs from Arabidopsis proteins is shown in FIG. 2. TPR1 of maize NPG1 showed 63% identity and 69% similarity with TPR1 of AtNPG1. TPR2 of maize NPG1 showed 43% identity and 59% similarity with TPR2 of AtNPG2. TPR3 of maize NPG1 had 79% identity and 85% similarity with TPR2 of AtNPG1. A BLAST search also revealed sequence similarities between maize NPG1 TPRs and TPRs from a variety of other proteins.

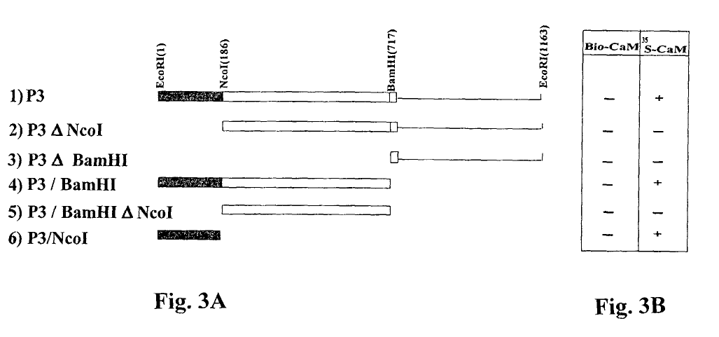

The maize protein encoded by the partial cDNA (1.2 kb) showed CaM-binding, suggesting that the CaM-binding domain is present in the region between 418 and 659 amino acids. To map the location of the CaM-binding domain, four truncated versions of the cDNA were made (FIG. 3) and expressed in E. coli as His-Tag fusions using pET28 expression vector. Protein gels of the fusion proteins were stained with Coomassie and the corresponding blots detected with various probes. The presence of the fusion protein was detected with T7 tag antibody whereas the binding of the fusion protein to CaM was tested with either radiolabeled or biotinylated CaM as described in the Examples Section.

The expression of the expected size fusion proteins was verified by probing the blot with T7 tag antibody. Probing a duplicate blot with ³⁵S-CaM showed that the protein (16 kDa) expressed from the N-terminal 168-bp region of the cDNA contained the putative CaM-binding domain. Truncated clones P3/BamHI and P3/NcoI which included the 168-bp region of the cDNA also produced CaM-binding peptide. Therefore, the protein encoded by the 168-bp truncated cDNA contained the CaM-binding domain.

The expressed protein was mainly detected in insoluble inclusion bodies although under some conditions a small amount was expressed in soluble cell fractions. No binding to CaM was observed in the presence of EGTA, confirming the Ca²⁺ dependency of the binding of the NPG1 polypeptide to CaM. The protein did not bind to biotinylated CaM when a duplicate blot was probed with biotinylated CaM in the presence of Ca²⁺. This is probably related to the biotin moiety on CaM that may have interfered with its binding to NPG1 protein. The protein expressed from clone 1 (P3) was solubilized in 6 M urea and successfully purified on a bovine CaM-Sepharose affinity column, further confirming the binding of the NPG1 protein to CaM.

In many CaM target proteins in animals, the CaM-binding domain has been shown to reside in a stretch of 18-20 amino acid residues (Snedden and Fromm (1998) Trends Plant Sci. 3:299-304; Zielinski, R. E. (1998) Annu. Rev. Plant Physiol. Plant Mo. Biol. 49:697-725; Reddy and Reddy (2002) Handbook of Plant and Crop Physiology (Ressarakli, M., ed) Marcel Dekker Inc., New York, 697-732). Although the amino acid sequence in the CaM-binding domain of different CaM target proteins is not conserved, the binding region is predicted to form a basic, amphiphilic α-helix in which hydrophobic residues are segregated from hydrophilic residues along the helix (O'Neil and DeGrado (1990) Trends Biochem. Sci. 56:319-327). Studies using synthetic peptides confirmed the speculation that CaM recognizes amphiphilic peptides (O'Neil and DeGrado (1990) supra).

CaM binding studies with truncated proteins of maize NPG1 have shown that the CaM-binding domain is located in a 56-aa stretch (residues 418-474 in FIG. 1). Analysis of this stretch of amino acids using a helical wheel program revealed a region from 421 to 438 that forms a basic amphiphilic α-helical structure. To test if this 18-aa stretch binds CaM, a synthetic peptide containing these amino acids was synthesized and used for binding studies. The synthetic peptide (MP-1, VSKGWRLLALILSAQQRF, SEQ ID NO:2) bound to bovine CaM and to CaM isoforms 2, 4, and 6 from Arabidopsis at concentrations as low as 0.5 μg, show that this region is involved in CaM binding.

Another synthetic peptide (MP2, AKLDQGSLL-RVKAKLKVAQSSPM, SEQ ID NO:5) corresponding to a different region of maize NPG1 polypeptide but lacking typical features of CaM-binding domains was used as a negative control in the binding studies. Neither MP2 nor BSA showed any binding to the labeled CaM isoforms. These results showed the specificity of the synthetic peptide MP-1 in binding to CaM. A previously characterized CaM-binding peptide of a microtubule motor protein (KCBP, SEQ ID NO:6) bound to all three CaM isoforms (Reddy et al. (1996) J. Biol. Chem. 271:7052-7060).

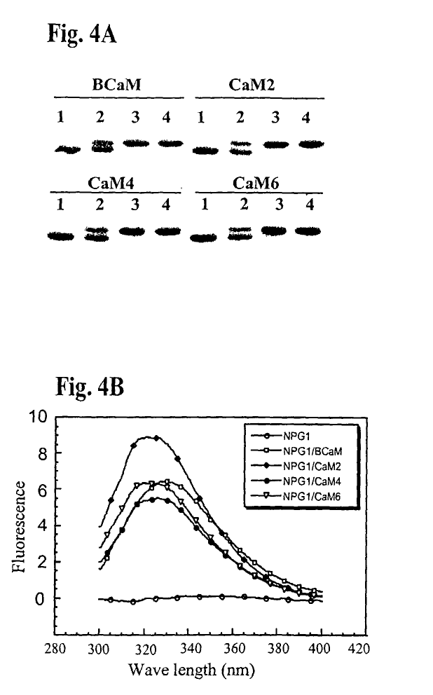

To determine the stoichiometry of the CaM-peptide complex, we performed binding studies using the synthetic peptide (MP-1) and CaM in the presence of Ca²⁺ or EGTA. The binding of the synthetic peptide to CaM was detected by a gel mobility shift assay in polyacrylamide gels containing 4 M urea (FIG. 4A). At 4 M urea, low affinity and nonspecific complexes dissociate while high affinity complexes remained intact. In the presence of CaCl₂ the synthetic peptide retarded the migration of CaM in the gel indicating the formation of a complex between the peptide and CaM (FIG. 4A). No change in CaM mobility was observed in the presence of EGTA indicating that Ca²⁺ is required for the formation of CaM-peptide complex. At a molar ratio of 1:1 (peptide:CaM), about 50% of CaM showed a shift (FIG. 4A, lane 2). At a molar ratio of 2:1 and 4:1 (peptide:CaM), the entire CaM migrated as a complex, and the band corresponding to the free CaM disappeared (FIG. 4A, lanes 3 and 4).

The synthetic peptide retarded mobility of CaM similarly with bovine CaM and the three AtCaM isoforms, indicating that the peptide binds to these CaMs in the same stoichiometry (FIG. 4A). Peptide-CaM complexes that do not dissociate in 4 M urea have dissociation constants of less than 100 nM (Erickson-Vitanen and DeGrado (1987) Methods Enzymol. 139:455-478). These mobility assays suggest that the binding between the peptide and CaM is strong and does not dissociate in the presence of 4 M urea.

The binding of a peptide to CaM can also be tested by fluorescence spectroscopy since the peptide contains a tryptophan residue that is absent in CaM. Tryptophan-containing peptides have been shown to, upon binding to CaM, shift their fluorescence spectrum and change the intensity of fluorescence (Reddy et al. (1996) supra; Erickson-Vitanen and DeGrado (1987) supra; Malencik and Anderson (1983) Biochem. Cell Biol. 114:50-56; Malencik and Anderson (1984) Biochemistry 23:2420-2428; Lukas et al. (1986) supra). In this example, the MP1 peptide contained a tryptophan and therefore was tested for fluorescence shift at equimolar ratios of peptide and CaM. As shown in FIG. 4B, the fluorescence of the synthetic peptide was shifted significantly in the presence of bovine CaM and the three isoforms of At-CaMs. The wavelength of peak emission shifted from about 350 to about 315 nm. In addition, the fluorescence intensity increased from about 0.1 to about 4.5, 6.1, and 8.4 in the presence of CaM4, bovine CaM/CaM6, and CaM2, respectively. The difference among the fluorescence intensity shifts of the different CaM isoform-peptide complexes indicates variation in the affinity of the CaM isoforms to various maize NPG1 peptides.

To determine the expression of NPG1 in maize, total RNA from maize roots, shoots, kernels, and pollen was isolated and an RNA gel blot analysis was performed. A single transcript of about 2 kb was detected only in the pollen and the germinated pollen. The transcript was absent in all other tissues. These results were further confirmed by reverse transcription-polymerase chain reaction where the NPG1 transcript was not detected in maize roots, shoots, and kernels. To demonstrate the presence of first strand cDNA in reverse transcriptase-polymerase chain reaction, another maize gene (CBP-1) that is expressed in root, shoot, and kernel was amplified (Reddy et al. (1993) Plant Sci. 94:109-117). The CBP-1 transcript was detected in all of the above tissues. Northern and reverse transcriptase-polymerase chain reaction results indicated that NPG1 is expressed only in pollen. Southern analysis of maize DNA digested with different restriction enzymes, revealed a single hybridizing band, indicating that NPG1 in maize is a single copy gene.

To determine the expression of NPG1 and the native size of the protein, total protein was extracted from maize roots, leaves, shoot tips, and pollen tissues, separated on a SDS-polyacrylamide gel electrophoresis, blotted onto polyvinylidene difluoride membranes, and NPG1 was detected using affinity-purified NPG1 antibody. A band of about 72 kDa was detected only in pollen and was absent in all other maize tissues, indicating pollen-specific expression of NPG1. The size of the immunoreactive protein was the same as the predicted size for the NPG1 gene. Two faint smaller size bands on immunoblots were most likely degradation products of NPG1 polypeptide as the intensity of those bands varied depending on the presence or absence of protease inhibitors in the extraction buffer. The protein was found in both the soluble and the microsomal fractions of the pollen.

To examine the presence of the protein during germination and growth of the pollen tubes, maize pollen was germinated on media for 30, 60, 120, and 240 minutes. Protein extracts from germinated pollen were separated and detected as described above. The level of NPG1 protein was fairly constant throughout germination. There was some decline, however, in the amount of NPG1 polypeptide after 30 minutes. This suggests that the protein is stored in the mature pollen and may be depleted in the first half hour of germination and is resynthesized during germination and pollen tube growth. These results indicate that this protein is expressed in mature pollen and during germination, suggesting a role for NPG1 in pollen germination and tube growth.

Studies with bacterially expressed fusion proteins indicate that the truncated C-terminal region of NPG1 binds to CaM in the presence of Ca²⁺. However, these results did not show that the full-length NPG1 polypeptide binds CaM. To demonstrate that the native NPG1 protein interacts with CaM in a Ca²⁺-dependent manner, NPG1 protein was isolated from maize pollen either by using a pull-down assay with CaM-Sepharose beads or by passing protein through a CaM-Sepharose column. The proteins that were isolated with both of these methods were blotted and probed with either affinity purified NPG1-specific antibody or HRP-CaM. Pollen CaM-binding proteins bound to CaM-Sepharose column in the presence of Ca²⁺ were eluted in a buffer containing EGTA. The spectral curves at 235 and 280 nm clearly showed the elution of CaM-binding proteins with EGTA.

The initial soluble protein extract, the flow-through, the wash, and the eluted proteins from the column were analyzed as well as the pull-down assay with CaM-Sepharose beads. These proteins were separated along with the bacterially expressed truncated NPG1 peptide (P3) and Arabidopsis KCBP 1.5C (Song et al. (1997) Proc. Natl. Acad. Sci. US.A. 94:322-327) on three gels. One gel was stained with Coomassie Blue. The other two were blotted onto nitrocellulose membranes. One blot was probed with NPG1-specific antibody to detect NPG1 peptide and the second one was subjected to HRP-CaM overlay assay to detect CaM-binding proteins. The majority of the pollen proteins did not bind to the CaM-Sepharose column. NPG1-specific antibody detected a single band (72 kDa) protein in crude extract and EGTA eluted fraction. Detection of NPG1 only in EGTA eluted fraction and not in either flow-through or wash fractions by NPG1-specific antibody and CaM overlay assay showed that native NPG1 binds CaM in the presence of Ca²⁺.

The positive control, P3, was also immunodetected by NPG1-specific antibody. However, HRP-CaM detected several other unknown CaM-binding proteins in both crude extract and elution fractions from CaM-Sepharose. No CaM-binding proteins were detected in flow-through and wash fractions, suggesting that all CaM-binding proteins in the pollen extract were bound to CaM-Sepharose and eluted with EGTA. KCBP, a well characterized CaM-binding protein from Arabidopsis was used as another positive control. KCBP was detected by HRP-CaM but not by NPG1-specific antibody.

Since several proteins were bound to CaM-Sepharose, it is possible that NPG1 polypeptide binds CaM-Sepharose indirectly through other proteins bound to the column. However, two lines of evidence eliminate this possibility. First, the protein detected by NPG1 specific antibody was also detected by HRP-CaM in a blot overlay assay. Second, bacterially expressed truncated NPG1 peptide bound to CaM-Sepharose in a Ca²⁺-dependent manner. These results clearly showed that the native NPG1 binds CaM in the presence of Ca²⁺.

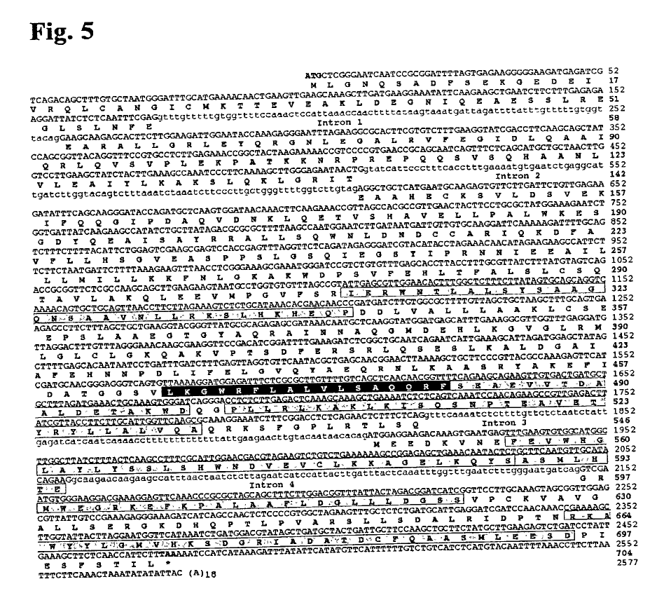

To understand the role of CaM-binding proteins in pollen germination and tube growth, we identified a homologue of a maize CaM-binding protein, NPG1, in the recently completed Arabidopsis genome sequence. We cloned the corresponding cDNA from Arabidopsis pollen RNA by reverse transcription-polymerase chain reaction (RT-PCR) using the primers corresponding to the 5′ and 3′ ends of the gene as described in Example 21. An expected size fragment (2.3 kb) was amplified by RT-PCR. Amplification from genomic DNA with the corresponding primers yielded a larger size (˜2.6 kb) product due to introns. The identity of the PCR-amplified products was confirmed by probing the blotted gels with ³²P labeled maize NPG1 cDNA. Comparison of the NPG1 cDNA sequence with the genomic sequence has revealed the presence of four introns in the coding region of the NPG1 gene (FIG. 5). The Arabidopsis NPG1 (AtNPG1) encodes a protein of 704 amino acids (FIG. 5, SEQ ID NO: 10). The deduced amino acid sequence of Arabidopsis NPG1 (Genbank accession number AF474176) differs from the predicted protein in the Arabidopsis genome database at the C-terminus due to inaccurate prediction of the last two introns. The Arabidopsis NPG1 amino acid sequence showed 52% identity and 60% similarity to maize NPG1 polypeptide. Analysis of the predicted amino acid sequence of Arabidopsis NPG1 using InterPro scan (Apweiler et al. (2001) Nuc. Acid Res. 29:37-40) revealed the presence of six tetratricopeptide repeats (TPRs) (FIG. 5). The TPR domains consist of a degenerate 34 amino acid region and are implicated in protein-protein interaction (Lamb et al. (1995) Trends Biochem. Sci. 20:257-259; Tzamarias and Struhl (1995) Genes & Dev. 9:821-831). The second TPR overlaps with the calmodulin-binding domain (FIG. 5). A homolog of NPG1 has not been found in any non-plant systems including yeast, fly (D. melanogaster), worm (C. elegans) and human (H.sapiens) genomes whose sequences have been completed, suggesting that it is a plant-specific CaM-binding protein.

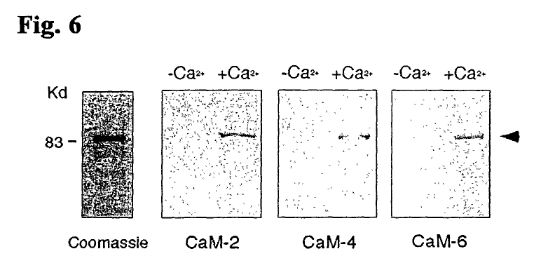

To demonstrate that AtNPG1 is a CaM-binding protein, the coding region of NPG1 was expressed as a T7 tag fusion using an E. coli expression system and used the bacterially expressed protein in CaM-binding studies. The bacterially expressed AtNPG1 bound CaM-Sepharose beads in a calcium-dependent manner and eluted with EGTA-containing buffer. The purified AtNPG1 bound three Arabidopsis CaM isoforms in a calcium-dependent manner in blot overlay assays (FIG. 6). These results confirm that AtNPG1 is a CaM-binding protein. The interaction of NPG1 with CaM only in the presence of calcium suggests that the function of NPG1 is modulated in vivo by calcium and CaM. To map the CaM-binding domain, a truncated version (aa 448-496 of SEQ ID NO:10) of Arabidopsis NPG1 which contains a putative calmodulin-binding domain was expressed in E. coli and tested for its ability to bind CaM. The truncated protein bound CaM in a manner similar to the full-length protein, indicating that the CaM-binding domain (CBD) is located between amino acids 448 and 496. The CBD was further mapped to an eighteen amino acid stretch (aa 464-481) by using synthetic peptides in a gel-shift assay.

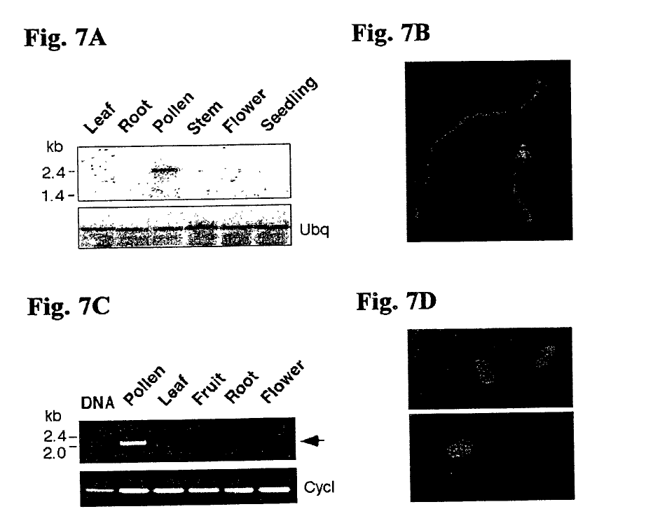

To determine the expression of Arabidopsis NPG1 gene, RNA from different tissues/organs was analyzed by Northern analysis. As shown in FIG. 7A, NPG1 transcripts were detected only in pollen. The transcript size (˜2.5 kb) is consistent with the expected size. Equal amounts of RNA on the blot were verified by hybridizing the blot with ubiquitin (FIG. 7A, lower panel). To eliminate the possibility that NPG1 may be expressed at very low levels, we performed reverse transcription-PCR (RT-PCR) analysis. The results from the RNA blot analysis were confirmed by RT-PCR where the NPG1 transcript was detected only in pollen (FIG. 7B). The amplified transcript was verified by hybridization with AtNPG1 cDNA as probe. Equal amounts of cDNA template in RT-PCR assays were verified by amplifying cyclophilin, a constitutively expressed gene. We have further confirmed pollen-specific expression of NPG1 by fusing its promoter to green fluorescent protein (GFP) and analyzing the promoter activity. In transient expression assays with tobacco pollen a very strong expression of GFP was found in pollen tubes (FIG. 7C). In transgenic Arabidopsis plants GFP was seen in mature and germinating pollen (FIG. 7D). The earliest expression of GFP was detectable in pollen at stages 12 to 13 of flower development (Smyth et al. (1990) Plant Cell 2:755-767). GFP was not detected in any other tissues except pollen. These results clearly show that NPG1 is expressed only in pollen and pollen tubes.

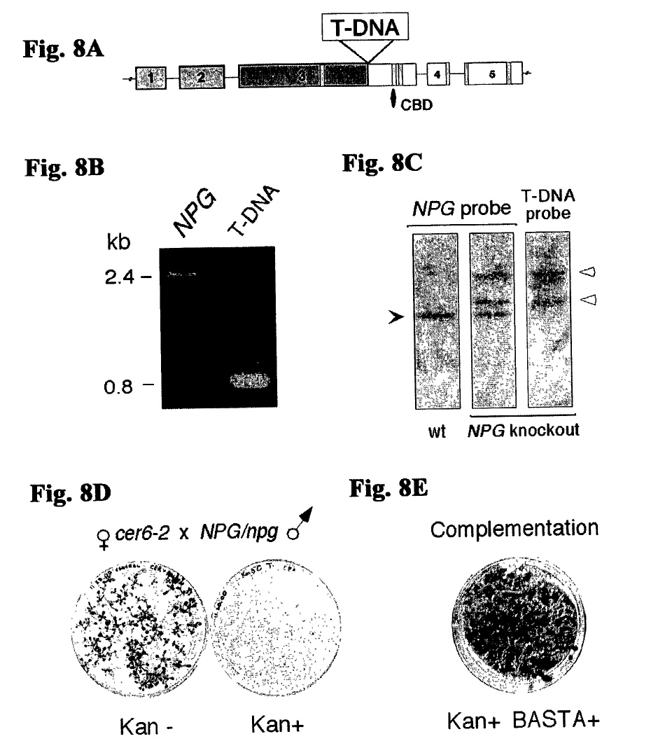

Since NPG1 is expressed exclusively in pollen it is likely to function in one or more of the following processes: pollen development, germination and/or pollen tube growth. To analyze the function of NPG1, a knockout mutant was isolated by screening a mutant library (60,400 independent kanamycin-resistant lines), which was generated by T-DNA insertion, using a reverse genetics screen (Krysan et al. (1999) Plant Cell 11:2283-2290; Sussman et al. (2000) Plant Physiol. 124:1465-1467). A “reverse genetics” screen uses a mutant library generated by T-DNA insertional mutagenesis and RT-PCR to identify mutants in a gene of interest based on gene sequence. A plant with a T-DNA insertion in NPG1 was identified by PCR amplification of a 0.8 kb product by a reverse primer of NPG1 and T-DNA left border primer (FIGS. 8A and 8B). The details of these analyses are provided in Example 24 below. Sequence analysis of the PCR product amplified from the insertion allele revealed that the T-DNA interrupted the coding region after 461 amino acids in exon 3 (FIG. 8A). Any protein product from this allele would lack the CBD and five putative TPRs (FIG. 8A) that are known to be involved in protein-protein interaction. Amplification of both the wild-type gene (with a gene specific primer set) and the mutated gene (with a T-DNA left border primer and a gene-specific reverse primer) from the plant suggested that it was a heterozygous plant (FIG. 8B). To confirm that the mutant plant is heterozygous and to determine the number of T-DNA insertions, DNA was isolated from wild type and mutant, and digested with a restriction enzyme (Bam HI) which does not cut NPG1 but has a single site in T-DNA. Therefore, it is expected that a Southern Blot probed with NPG1 probe should produce a single hybridizing band in wild type and three bands in the mutant. Furthermore, if there is a single insertion, the Southern Blot with DNA from the mutant probed with the T-DNA should produce two hybridizing bands. As shown in FIG. 8C, Southern analysis with NPG1 and T-DNA probes confirmed that the plant was heterozygous with a single T-DNA insertion in the mutant allele (FIG. 8C).

Heterozygous mutant plants were selfed to obtain homozygous plants. Analysis of the progeny from this cross revealed two significant findings. First, the ratio of kanamycin resistant to kanamycin sensitive plants was 1:1 (476:444) instead of the expected 3:1. Second, Southern and PCR analyses of the kanamycin resistant progeny with primers that amplify the wild type and mutant allele showed that all kanamycin resistant plants were heterozygous. A distorted segregation ratio and an inability to produce homozygous plants, together with pollen-specific expression of NPG1, suggested that the mutated gene is not transmitted through the male gametophyte.

To further confirm that the npg1 allele is not transmitted through pollen, we used a convenient male sterile mutant (cer6-2) (Preuss et al. (1993) Genes & Dev. 7:974-985) to cross with an NPG1/npg1 plant (FIG. 8D). As expected, we did not obtain any kanamycin resistant progeny from this cross (FIG. 8D). However, pollination of emasculated NPG1/npg1 flowers with pollen from wild type plants produced the expected 1:1 kanamycin resistant:sensitive progeny demonstrating that transmission of the npg1 allele through female gametes is not affected. These results confirm that the mutant allele is not transmitted through pollen.

To demonstrate that the mutant phenotype is due to disruption of the NPG1 gene, NPG1/npg1 plants were transformed with an NPG1 cDNA driven by the NPG1 promoter (FIG. 8C) in a vector that confers resistance to BASTA (Crescent Chemical, Islandia, N.Y.). Plants resistant to both kanamycin and BASTA were selected. Pollen from 48 kanamycin and BASTA resistant plants was used to pollinate a male sterile (cer6-2) mutant (Preuss et al. (1993) supra) to test transmission of npg1 allele. From this cross, we obtained progeny that is resistant to both selective agents from 37 out of 48 total crosses (an example is shown in FIG. 8E). The presence of introduced cDNA in the kanamycin and BASTA resistant progeny was verified by amplifying the cDNA from genomic DNA. These results provide evidence that expression of the introduced NPG1 cDNA complements the mutant NPG1 allele and that the mutant phenotype is indeed due to the T-DNA insertion in the NPG1 gene.

Because the npg1 allele is not transmitted through pollen we envisioned two possible scenarios by which the mutated allele (npg1) is not transmitted through pollen. One is that haploid cells that inherit the mutant allele after meiosis (half of the haploid cells) do not develop into pollen. Alternatively, the haploid cells carrying npg1 could develop normally but not be functional due to a defect in later events of pollen germination and/or tube growth.

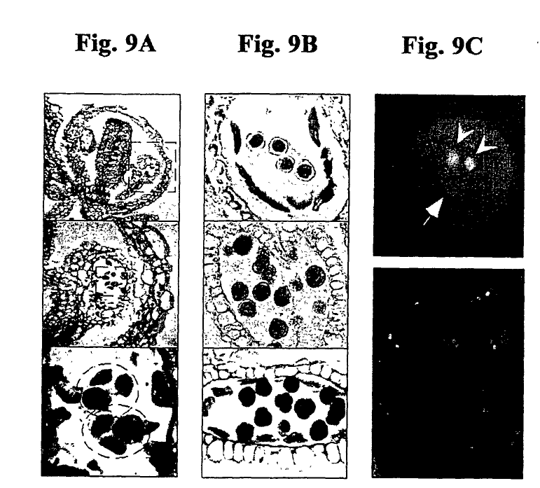

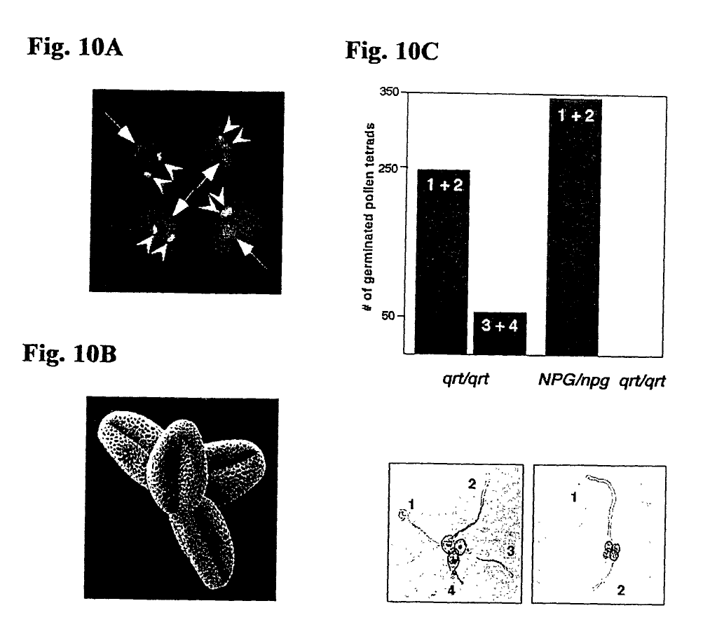

To address the first possibility, we analyzed the pollen development in wild type and NPG1/npg1 plants by light microscopy. We found that in the mutant, normal tetrads are formed after meiosis and the microspores developed into morphologically normal pollen (FIGS. 9A and 9B) (Sander et al. (1999) Sex. Plant Reprod. 11:297-322). Pollen from NPG1/npg1 plants was indistinguishable from wild type in their shape, size and DAPI staining, which showed all three nuclei (a single diffusely staining vegetative nucleus and two condensed sperm nuclei per pollen) (FIG. 9C). These results indicate that pollen development is normal in NPG1/npg1 plants. To further confirm these results we used the quartet1 (qrt/qrt) mutant of Arabidopsis. In the qrt1 mutant the four microspores/pollen grains produced after male meiosis remain attached as tetrads due to fusion of the outer walls of the four meiotic products of pollen mother cell (Preuss et al. (1994) Science 264:1458-1460). Therefore, any abnormalities in pollen development can be determined by analyzing the tetrads (Preuss et al. (1993) supra). We generated a double mutant (NPG1/npg1, qrt/qrt) as described in the Examples Section. Based on our earlier microscopic observations we expected to see normal development of all four pollen grains including the ones that carry the npg1 allele in the double mutant. The tetrads in the double mutant were normal in appearance and were indistinguishable from tetrads of the qrt mutant (FIGS. 10A and 10B). Furthermore, DAPI staining revealed the presence of the vegetative and two sperm nuclei in each pollen grain in the tetrad (FIG. 10A). Scanning electron micrographs of tetrads from the double mutant showed that all four pollen grains in a tetrad are indistinguishable in their morphology (FIG. 10B). These studies clearly indicate that NPG1 is not essential for male meiosis and pollen development.

To address the role of NPG1 in pollen germination and/or tube growth, we used the tetrads from the double mutant (NPG1/npg1, qrt/qrt) and qrt/qrt flowers (Smyth et al. (1990) supra) for in vitro germination (Fan et al. (2001) J. Exp. Bot. 52:1603-1614). If pollen germination is affected in the double mutant we expected to see germination of no more than two of the four pollen grains in a tetrad whereas up to four pollen grains can germinate from qrt/qrt plants. The germination of pollen in tetrads from the qrt/qrt mutant ranged from one to four whereas only one or two pollen grains germinated from tetrads from the double mutant (FIG. 10C). One out of five germinating tetrads from qrt/qrt plants showed germination of three or four pollen grains. In contrast, all germinating tetrads from the double mutant showed germination of only one or two pollen grains (FIG. 10C). These results, together with no homozygous mutant and kanamycin resistant plants obtained from a cross between a male sterile mutant (cer6-2) and NPG1/npg1 mutant, indicate that pollen carrying the npg1 allele does not germinate and NPG1 is a key regulator of pollen germination.

Many sporophytic mutants that affect microsporogenesis have been isolated by screening for male sterility (Preuss et al.(1993) supra; Sander et al. (1999) supra). In contrast, isolation of gametophytic mutants affecting pollen development and/or function has been difficult for obvious reasons. Such mutants as heterozygotes produce 50% normal pollen, and thus show no easily observable affect on fertility. Further, it would not be possible to obtain homozygous mutant plants. So far, a few male gametophytic mutants that affect pollen development and/or function have been identified by screening T-DNA insertion lines for distorted segregation ratios of antibiotic resistance or by screening mutant populations for abnormal pollen (Chen and McCormick (1996) Development 122:3242-3253; Howden et al. (1998) Genetics 149:621-631; Park et al. (1998) Development 125:3789-3799; Procissi et al. (2001) Genetics Sander et al. (1999) 158:1773-1783). To our knowledge, npg1 is the first gametophytic mutant for which the gene is cloned and the encoded protein is known to be required for pollen germination.

The studies described above clearly indicate that NPG1, a calmodulin binding protein, is required for pollen germination and is likely to be a key protein in the calcium signaling during pollen germination and tip growth. The nucleotide and amino acid sequences for NPG1 provided in the present application are useful in generating male sterile plants and identifying other proteins that are involved in the calcium signaling by binding to NPG1.

The fact that the multiple TPR motifs are present in the NPG1 polypeptide raises the possibility that there are additional proteins that interact with NPG1. A person of ordinary skill in the art can employ the yeast two-hybrid system (Fields and Song (1989) Nature 340:245-246) to clone cDNAs encoding proteins that interact with NPG1. This approach takes advantage of the modular domain structure of eukaryotic transcription factors. The transcription factor that is used in this approach has an amino-terminal domain that binds to specific DNA sequences and a C-terminal domain that is necessary for activation of transcription. Neither domain alone will activate transcription. It has been shown that the DNA binding domain does not have to be physically on the same polypeptide as the activation domain (Field and Song (1989) supra). Transcription can be used to assay the interaction between two proteins if one was fused to the DNA-binding domain and the other was fused to the activation domain. The yeast two-hybrid approach was used to isolate four novel splicing factors that interact with Arabidopsis U1 snRNP 70K protein (Golovkin and Reddy (1998) Plant Cell 10:1637-1647; Golovkin and Reddy (1999) J. biol. Chem. 36428-36483, 36428-36438) and a protein that interacts with a molecular motor (Day et al. (2000) J. Biol. Chem. 275:13737-13745).

In order to identify a protein(s) that interact with NPG1, AtNPG1 cDNA has been cloned in bait vector (pAS). Since AtNPG1 is expressed only in pollen, a cDNA library is prepared in prey (pACT vector) expression plasmid of the yeast two hybrid system using RNA isolated from pollen. Yeast strain, Y190, which allows dual selection, is used to screen the cDNA libraries to isolate clones interacting with AtNPG1. This strain has two reporter genes, a LacZ gene and a selectable HIS3 gene, under the control of a GAL1 promoter, which is activated by GAL4. Low expression of HIS3 is required for prototrophy, which makes the screening more sensitive and allows detection of weak interaction between hybrid proteins.

First, Y190 strain is transformed with pAS-AtNPG1 construct and the transformed cells are assayed for β-galactosidase activity. If the yeast cells containing the pAS-AtNPG1 are negative for β-galactosidase activity, then the cells containing pAS-AtNPG1 are transformed with a library that is prepared in pACT vector. Transformants that are His+ prototrophs are further screened for β-galactosidase activity, which eliminates His+ revertants and false positives (Durfee et al. (1993) Genes & Development 7:555-569). Colonies that are His+ and are blue (β-gal positives) are used for further analysis. The plasmids from the positives are isolated and retransformed into Y190 in the presence and absence of bait plasmid to confirm the interaction between AtNPG1 and the protein coded by the positive clone. The details of this technology can be found in Fields and Song (1989) supra.

Proteins that bind NPG1 can also be identified by screening expression libraries with bacterially expressed NPG1 peptides. This method has been used to isolate several novel calmodulin-binding proteins from plants (Fordham-Skelton et al. (1994) Plant Mo. Bio. Reporter 12:355-363; Reddy et al. (1996a) J. Biol. Chem. 271:7052-7060; Reddy et al. (1996b) Plant J. 10:9-21). In initial studies, protein blots are prepared with pollen extracts and probed with labeled or tagged AtNPG1 (e.g., S.tag fused AtNPG1) to determine if there are any protein(s) that interact with AtNPG1. S protein is used to identify the peptides that interact with AtNPG1. If the initial studies show an indication that there is a protein that interacts with AtNPG1, an expression library prepared from RNA of pollen tissue can be screened to obtain the clone encoding such protein. Purified tagged AtNPG1 is needed to screen an expression library with AtNPG1. Full-length Arabidopsis AtNPG1 cDNA is cloned into pET 32 (Studier et al. (1990) Methods Enzymol. 185:60-89; Narasimhulu and Reddy (1998) Plant Cell 10:957-965). The fusion protein contains an S.Tag sequence and a stretch of six consecutive histidine residues. (Hochuli et al. (1987) J. Chromatography 411:177-184; Golovkin and Reddy (1999) supra). The S.tag allows the detection of these proteins using S protein. The purified AtNPG1 fusion protein is used for screening Arabidopsis expression libraries.

A majority of the NPG1 peptides disclosed in the present application was prepared by employing recombinant DNA technology well known in the art. However, because of their relatively small size, the peptides of the invention can also be synthesized in solution or on a solid support in accordance with conventional techniques. Various automatic synthesizers are commercially available and can be used in accordance with known protocols. See, for example, Stewart and Young (1984) Solid Phase Peptide Synthesis, 2d. ed., Pierce Chemical Co.; Tam et al. (1983) J. Am. Chem. Soc. 105:6442; Merrifield (1986) Science 232:341-347; and Barany and Merrifield (1979) The Peptides, Gross and Meienhofer, eds., Academic Press, New York pp. 1-284, each incorporated herein by reference. Short peptide sequences, or libraries of overlapping peptides, usually from about 6 up to about 35 to 50 amino acids, which correspond to selected regions of the NPG1 protein, such as SEQ ID NO:2, can be readily synthesized and then screened in screening assays designed to identify reactive peptides.

The present invention also provides for the use of NPG1 proteins or peptides as antigens for the immunization of animals to produce NPG1-specific polyclonal as well as monoclonal antibodies. Means for preparing and characterizing antibodies are well known in the art (see, e.g., Harlow and Lane (1988), Antibodies: A Laboratory Manual, Cold Spring Harbor Laboratories, Cold Spring Harbor, N.Y.; Goding (1986) In Monoclonal Antibodies: Principles and Practice, 2d ed., Academic Press, Orlando, Fla., pp. 60-61, and 71-74; and Ausubel et al. (1993) Current Protocols in Molecular Biology, Wiley Interscience/Greene Publishing, New York, N.Y.).

The finding that the nucleotide and amino acid sequences of maize and Arabidopsis NPG1 show high degree of homology suggest that there is likely a NPG1 homolog in other plants. A person of ordinary skill in the art can identify such homologs by using the nucleic acid sequence of maize or Arabidopsis NPG1 gene or fragments thereof as probe. Suitable hybridization conditions for such experimentation are well known to those of skill in the art. Stringency can be controlled by temperature, probe concentration, probe length, ionic strength, time, and the like. Preferably, hybridization is conducted under moderate to high stringency conditions by techniques well know in the art, as described, for example in Keller, G. H., M. M. Manak (1987) DNA Probes, Stockton Press, New York, N.Y., pp. 169-170, hereby incorporated by reference. For example, a medium stringency condition could be provided by about 0.1 to 0.25 M NaCl, at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results. Another example of high stringency conditions are hybridizing at 68° C. in 5× SSC/5× Denhardt's solution/0.1% SDS, and washing in 0.2× SSC/0.1% SDS at room temperature. An example of conditions of moderate stringency are hybridizing at 68° C. in 5× SSC/5× Denhardt's solution/0.1% SDS and washing at 42° C. in 3× SSC. The parameters of temperature and salt concentration can be varied to achieve the desired level of sequence identity between probe and target nucleic acid. See, e.g., Sambrook et al. (1989) vide infra or Ausubel et al. (1995) Current Protocols in Molecular Biology, John Wiley & Sons, NY, N.Y., for further guidance on hybridization conditions.

For double-stranded DNA gene probes, hybridization can be carried out overnight at 20-25° C. below the melting temperature (Tm) of the DNA hybrid in 6× SSPE 5× Denhardt's solution, 0.1% SDS, 0.1 mg/ml denatured DNA. The melting temperature is described by the following formula (Beltz, G. A., Jacobe, T. H., Rickbush, P. T., Chorbas, and F. C. Kafatos (1983) Methods of Enzymology, Wu, Grossman, and Moldave [eds.] Academic Press, New York 100:266-285).

Tm=81.5° C.+16.6 Log [Na+]+0.41(+G+C)−0.61(%formamide)−600/length of duplex in base pairs.

Washes are typically carried out as follows: twice at room temperature for 15 minutes 1× SSPE, 0.1% SDS (low stringency wash), and once at the hybridization temperature for 15 minutes in 1× SSPE, 0.1% SDS (moderate stringency wash).

In general, salt and/or temperature can be altered to change stringency. With a labeled DNA fragment of >70 or so bases in length, the following conditions can be used: Low, 1 or 2× SSPE, room temperature; Low, 1 or 2× SSPE, 42° C.; Moderate, 0.2× or 1× SSPE, 65° C.; and High, 0.1× SSPE, 65° C.

Alternatively, NPG1 homologs in other plants can be isolated by any art-known methods including RT-PCR using a pair of primers derived from the conserved region of NPG1 or screening a pollen expression library using NPG1-specific antibodies that recognize an epitope in the conserved region. Polymerase Chain Reaction (PCR) is a repetitive, enzymatic, primed synthesis of a nucleic acid sequence. This procedure is well known and commonly used by those skilled in this art (see Mullis, U.S. Pat. Nos. 4,683,195, 4,683,202, and 4,800,159; Saiki et al. (1985) Science 230:1350-1354).

Silencing of a desired gene using antisense or double stranded inhibitor RNA (also known as hairpin RNA) has been widely used in plants (Wang et al. (2002) Curr. Opin. Plant Biol. 5:146-150; Wesley et al. (2001) Plant J. 27:581-590; Smith et al. (2000) Nature 407:319-320; Chuang et al. (2000) Proc. Natl. Acad. Sci. USA 97:4985-4990). The efficiency of these methods, especially with hairpin RNA, is shown to be high. Since NPG1 is found to be essential for pollen germination, it should be possible to block its expression to generate male sterile plants. Non-limiting examples of antisense technology for plant genetic engineering are disclosed in U.S. Pat. Nos. 6,172,279, 6,184,439, 6,211,437 and 6,215,045, the relevant portions of each of which are incorporated herein by reference. The NPG1 cDNA (full-length or fragments thereof) in the antisense orientation or in the form of hairpin fused to a strong promoter that is active in pollen mother cells as well as microspores can be introduced into plants by art-known plant transformation methods, for example, Agrobacterium-mediated transformation. Such a promoter will ensure that the antisense/hairpin RNA be expressed prior to and after male meiosis so that the all four products of male meiosis do contain the antisense/hairpin RNA and block expression of NPG1. It is known that transformation by Agrobacterium results in multiple insertions (up to ten or more). Such multiple insertions would ensure that all four products of male meiosis in at least some transgenic lines would contain and express the antisense/hairpin RNA. The transgenic lines in which NPG1 is blocked in all four pollen grains would be easy to identify based on their male sterile phenotype. Antisense constructs can also be designed to bind introns, or even exon-intron boundaries of the NPG1 gene.

Another approach for addressing plant male sterility is through the use of ribozymes. Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cech (1987) Proc. Natl. Acad. Sci USA 84:8788-8792; Gerlach et al. (1987) Nature (London) 328:802-805; Forster and Symons (1987) Cell 49:211-220).

It is anticipated that particularly appropriate targets for ribozyme or antisense directed induction of plant male sterility would be the genes or gene products for NPG1. A non-limiting example of the use of ribozyme technology in plant genetic engineering is disclosed in U.S. Pat. No. 6,215,045, the relevant portions of which are incorporated herein by reference.

The pollen specific transcription and translation regulatory elements provided in the present application (e.g., SEQ ID NO:3 and 4) are particularly useful for inhibiting allergen expression in pollen. Numerous studies report a variety of plant (Morgensen et al. (2002) J. Biol. Chem. April 12 in press; Modoro-Horiuti et al. (2001) 87(4): 261-71; Swoboda et al. (2002) 32(1):270-280; Luttkopf et al. (2002) 38(7):515-25) and the nucleotide sequences encoding several allergens are known in the art. Accordingly, those skilled in the art can prepare an expression vector containing an antisense nucleic acid of an allergen under the control of the maize or Arabidopsis pollen specific regulatory elements of the instant invention and produce a transgenic plant that shows less allergen production.

Methods of transforming plant cells that can be used within the scope of the present invention include microinjection (Crossway et al. (1986) Biotechniques 4:320-334), electroporation (Riggs et al. (1986) Proc. Natl. Acad. Sci. USA, 83:5602-5606), Agrobacterium mediated transformation (Hinchee et al. (1988) Biotechnology, 6:915-921), direct gene transfer (Paszkowski et al. (1984) EMBO J., 3:2717-2722), and biolistic particle acceleration (see, for example, Sanford et al., U.S. Pat. No. 4,945,050; and McCabe et al. (1988) Biotechnology, 6:923-926). Also see Sanford et al. (1987) Particulate Science and Technology, 5:27-37 (onion); Christou et al. (1988) Plant Physiol. 87:671-674 (soybean); McCabe et al. (1988) BiolTechnology, 6:923-926 (soybean); Datta et al. (1990) Biotechnology, 8:736-740 (rice); Gordon-Kamm et al. (1990) Plant Cell, 2:603-618 (maize); Kaeppler et al. (1990) Plant Cell Reports, 9:415-418; and Kaeppler et al. (1992) Theor. Appl. Genet., 84:560-566 (whisker-mediated transformation); D'Halluin et al. (1992) Plant Cell, 4:1495-1505 (electroporation); Li et al. (1993) Plant Cell Reports, 12:250-255 and Christou and Ford (1995) Annals of Botany, 75:407-413 (rice); Osjoda et al. (1996) Nature Biotechnology, 14:745-750 (maize via Agrobacterium tumefaciens); each of which is incorporated herein by reference.

The cells that have been transformed can be grown into plants in accordance with conventional ways. See, for example, McCormick et al. (1986) Plant Cell Reports, 5:81-84. These plants may then be grown, and either pollinated with the same transformed strain or different strains, and the resulting hybrid having the desired phenotypic characteristic identified. Two or more generations may be grown to ensure that the subject phenotypic characteristic is stably maintained and inherited and then seeds harvested for growing plants that possess the desired phenotype or trait. In certain plant species, for example corn, it may be desirable to engage in an extensive backcrossing program to ensure that transgenic seed, offered for commercial sale, contains a genetic background that is compatible with non-transgenic phenotypic characteristics, such as high yield, drought or disease resistance, early maturity, etc. The skilled artisan in plant breeding will be familiar with methods for crossing transgenic plants with non-transgenic stock to produce a transgenic progeny of the desired genetic background. In the present application, the term “progeny” refers to the offspring (children) of a particular cross, as well as all subsequent generations derived from that cross. It is generally considered that six or more generations of backcrossing may be desired to produce a progeny transgenic plant of appropriate genetic background.

Standard techniques for cloning, DNA isolation, amplification and purification, for enzymatic reactions involving DNA ligase, DNA polymerase, restriction endonucleases and the like, and various separation techniques are those known and commonly employed by those skilled in the art. A number of standard techniques are described in Sambrook et al. (1989) Molecular Cloning, Second Edition, Cold Spring Harbor Laboratory, Plainview, N.Y.; Maniatis et al. (1982) Molecular Cloning, Cold Spring Harbor Laboratory, Plainview, N.Y.; Wu (ed.) (1993) Meth. Enzymol. 218, Part I; Wu (ed.) (1979) Meth. Enzymol. 68; Wu et al. (eds.) (1983) Meth. Enzymol. 100 and 101; Grossman and Moldave (eds.) Meth. Enzymol. 65; Miller (ed.) (1972) Experiments in Molecular Genetics, Cold Spring Harbor Laboratory, Cold Spring Harbor, N.Y.; Old and Primrose (1981) Principles of Gene Manipulation, University of California Press, Berkley; Schleif and Wensink (1982) Practical Methods in Molecular Biology; Glover (ed.) (1985) DNA Cloning Vol. I and II, IRL Press, Oxford, UK; Hames and Higgins (eds.) (1985) Nucleic Acid Hybridization, IRL Press, Oxford, UK; Setlow and Hollaender (1979) Genetic Engineering: Principles and Methods, Vols. 1-4, Plenum Press, New York; and Ausubel et al. (1992) Current Protocols in Molecular Biology, Greene/Wiley, New York, N.Y. Abbreviations and nomenclature, where employed, are deemed standard in the field and commonly used in professional journals such as those cited herein.

All references cited in the present application are incorporated by reference herein to the extent that there is no inconsistency with the present disclosure.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials

Maize (Zea mays L.) inbred lines KYS and A632 seeds were germinated on moist filter paper, and the tissues (roots, hypocotyls, and leaves) were collected after 10 days of germination. Mature pollen was collected from tassels of field or greenhouse-grown maize. Freshly collected maize pollen was germinated on a medium containing 12% sucrose, 300 mg/liter CaCl₂, 100 mg/liter boric acid, and 0.7% agarose. Triton X-100-free nitrocellulose filter discs were obtained from Millipore (Bedford, Mass.). Easy tag ³⁵S-isotope labeling mixture was obtained from PerkinElmer Life Sciences (Boston, Mass.). Exassist helper phage and Escherichia coli SOLR cells were obtained from Stratagene (La Jolla, Calif.). Nitro blue tetrazolium, 5-bromo-4-chloro-3-indolyl phosphate, IPTG, and Trizol were obtained from Life Technologies, Inc. (Gaithersburg, Md.). pET vectors and E. coli strain BL21(DE3) were purchased from Novagen (Madison, Wis.). Gelatin and diaminobenzidine were obtained from Sigma (St. Louis, Mo.). Biotinylated CaM and bovine CaM were from Calbiochem-Novabiochem (San Diego, Calif.). Complete protease inhibitor mixture was from Roche Molecular Biochemicals (Roche Applied Science, Indianapolis, Ind.). Phenyl-Sepharose CL-4B, bovine CaM Sepharose-4B, and CNBr-activated Sepharose-4B were obtained from Amersham Pharmacia Biotech UK Ltd. (Buckinghamshire, UK). Vanadyl ribonucleoside RNase inhibitor was from New England BioLabs (Beverly, Mass.). Expression and purification of the carboxyl-terminal region of KCBP (1.4C) was described in Reddy et al. (1996) supra. All other chemicals were of reagent grade.

### Example 2

### Expression and Purification of Recombinant Arabidopsis thaliana CaM Isoforms

pET expression vectors containing CaM-2, -4, or -6 isoforms were obtained from Dr. Raymond Zielinski (Liao and Zielinski (1995) Methods Cell Biol. 49:487-500). The expected molecular weights for AtCaM2, -4, and -6 are 16,808, 16,824, and 16,822, respectively. The AtCaM isoforms were induced and purified according to Liao and Zielinski (1995) supra with some modifications. The E. coli BL21(DE3) cells containing the recombinant pET CaM expression clone were grown to A₆₀₀ of 0.6 and induced by 1 mM IPTG for 3 h at 37° C. in 1 liter of NZY medium containing 50 mg/ml ampicillin according to Fromm and Chua (1992) Plant Mol. Biol. Rep. 10:199-206.

The following procedures were performed at 4° C. The cells were harvested, washed in buffer A (50 mM Tris-HCl, pH 7.5), and resuspended in extraction buffer (Buffer A with 2 mM EDTA, 1 mM DTT, 200 mg/ml lysozyme, and complete protease inhibitor mixture). After treatment with DNase to remove DNA, the cell extract was clarified by centrifugation and the supernatant fraction was precipitated with 55% ammonium sulfate. The proteins in the supernatant were precipitated with 50% H₂SO₄ (pH 4) for 30 min with stirring. After centrifugation, the pellet was resuspended in buffer A containing 1 mM DTT, dialyzed first in distilled water, and then in buffer A containing 100 mM NaCl, 0.5 mM EGTA, and 1 mM DTT.

After adjusting CaCl₂ concentration to 5 mM, the protein was loaded onto a phenyl-Sepharose column CL-4B (10 ml bed volume) pre-equilibrated with buffer B (buffer A containing 0.1 mM CaCl₂ and 0.5 mM DTT). The column was washed with buffer B containing 5 mM NaCl and the Arabidopsis thaliana CaM protein (AtCaM) was eluted with elution buffer containing 50 mM Tris-HCl, pH 7.5, 1 mM EGTA, and 0.5 mM DTT. The eluates containing the proteins were dialyzed in water.

### Example 3

### Production and Purification of ³⁵S-labeled AtCaM Isoforms

³⁵S-labeled AtCaM isoforms were prepared and purified according to Fromm and Chua (1992) supra with slight modifications. Initially, the cells were grown in M9 medium with 10 g/liter tryptone and 50 mg/ml ampicillin overnight and then the cells were concentrated in 10 ml of M9 medium with ampicillin. One mM IPTG was added to the cells (0.6 A₆₀₀), 2 mCi of Easy tag ³⁵S-labeling mixture was added after 15 min, and the cultures were grown for 3 h at 37° C. The cells were pelleted, resuspended in 1.5 ml of buffer A, lysed with lysozyme (0.2 mg/ml), and DNase-treated (50 units) in the presence of 3 mM MgCl₂. After centrifuging the lysate at 30,000 rpm for 30 min, the supernatant was heated for 3 min at 90° C., centrifuged, and the resulting supernatant was used to purify the radiolabeled CaM on a 1-ml phenyl-Sepharose CL-4B column.

### Example 4

### Screening of Maize Pollen Expression Library with ³⁵S-CaM

A cDNA library from maize pollen constructed in the EcoRI site of Zap II vector was used for screening. About 120,000 recombinants were screened with a mixture of ³⁵S-labeled AtCaM isoforms 4 and 6. Approximately 9000 pfu per 15-cm plate were plated on NZCYM plates using E. coli XL1-blue MRA (Stratagene, La Jolla, Calif.) as the host strain. The plates were incubated at 42° C. until the plaques appeared, at which time the plates were overlaid with nitrocellulose filters that were previously soaked in 10 mM IPTG. Plaques were allowed to resume growth over-night at 37° C. The plates were then placed at 4° C. for 1 h. The nitrocellulose filters were removed and washed briefly in TBS (50 mM Tris-HCl, pH 7.5, 150 mM NaCl, 5 mM CaCl₂). The filters were blocked in TBS containing 1% nonfat milk for 15 min with gentle shaking at room temperature and then incubated in a mixture of ³⁵S-labeled AtCaM isoforms at 5 mg/ml for 12 h. The membranes were rinsed in TBS/Ca²⁺ (TBS containing 5 mM CaCl₂) three times for 5 min each and dried between 2 sheets of 3MM paper for 24 h before exposing to an X-ray film. The putative positive plaques were plaque-purified by two additional rounds of screening. The cDNA inserts from Zap II were excised in vivo in a plasmid form using the Exassist helper phage and by infecting E. coli SOLR cells with phage recombinant. The insert was excised by digesting the plasmid DNA with EcoRI. The positive clones were confirmed for Ca²⁺ dependent binding to CaM by expressing the purified clones as above, incubating the membrane in ³⁵S-CaM probe, and washing the membrane in TBS buffer containing either 2 mM Ca²⁺ or 5 mM EGTA, a Ca²⁺ chelator.

### Example 5

### Isolation of the Genomic Clone

A maize genomic library in EMBL3 (CLONTECH, Palo Alto, Calif.) was screened with a ³²P-labeled cDNA (1.2 kb) according to the standard Sambrook et al. (1989) In: Molecular Cloning: A Laboratory manual, 2^{nd} Ed., Cold Spring Harbor Laboratory press, Cold Spring Harbor, N.Y. About 650,000 recombinants were screened and about 20 positive clones were isolated after two rounds of screening. Restriction mapping and Southern analysis of the positive clones indicated that all of the isolated clones were derived from the same gene. The clone with the largest hybridizing fragment was sequenced by primer walking.

### Example 6

### DNA Sequencing and Analysis

Both strands of cDNAs were sequenced by dideoxynucleotide chain termination using double stranded DNA (Sambrook et al., 1989 supra). Sequence analysis was performed using MacVector and Sequencher programs. BLAST searches were performed at the National Center for Biotechnology Information web site.

### Example 7

### Construction and Expression of Truncated cDNAs in E. coli

The 1.2-kb cDNA insert (named P3) containing the coding region for 242 amino acids in the carboxyl-terminal region was cloned in-frame into a pET28b expression vector. For mapping the CaM-binding domain, several truncated constructs were prepared in a pET vector. The P3 cDNA in pET28 was digested with either NcoI or BamHI to release a 168- or 717-bp fragment corresponding to the 5′ end of the cDNA. The NcoI and BamHI fragments were cloned into pET28b vector digested with the respective enzymes to generate P3/NcoI and P3/BamHI clones. The P3 clones lacking either NcoI or BamHI fragments were religated to generate PΔNcoI and PΔBamHI expression clones. A 531 -bp fragment representing the BamHI fragment without the NcoI fragment was also cloned in pET28b vector. All the clones were introduced into E. coli BL21(DE3) and expressed.

Expression of the protein was achieved by growing the bacterial cells containing the subclones at 37° C. to an A₆₀₀ of 0.6, IPTG was then added to the cultures to a final concentration of 1 mM and the cells were allowed to grow for 3 more hours at 30° C. The cells from induced and un-induced cultures were collected, resuspended in lysis buffer (50 mM Tris-HCl, pH 8.0, 2 mM EDTA, 100 mM lysozyme, 0.1% Triton X-100), vortexed, and incubated on ice for 1 h. The mixture was then sonicated 3 times, 10 s each, using a Virsonic digital 475 ultrasonic cell disruptor (Virtis, N.Y.). The lysate was then centrifuged at 12,000×g for 20 min to separate the soluble (supernatant) from the insoluble (pellet) fractions. The pellet was dissolved in either 6 M urea containing buffer or sample buffer, and the supernatant and the pellet were electrophoresed on 12% SDS-polyacrylamide gels. The gels were blotted onto a nitrocellulose membrane using a Bio-Rad transfer cell.

Expressed proteins were detected by T7 tag monoclonal antibody conjugated to alkaline phosphatase (T7-AP) (Novagen, Madison, Wis.). Briefly, the blots were blocked for 2 h in TBST buffer (10 mM Tris-HCl, pH 8.0, 150 mM NaCl, 0.1% Tween-20) containing 3% gelatin. The blots were then incubated for 30 min in T7-AP (1:10,000 dilution) in TBST containing 1% gelatin, washed three times with TBST, rinsed briefly in AP buffer (50 mM Tris-HCl, pH 9.5, 100 mM NaCl, 1 mM MgCl₂ ), and then developed in AP buffer containing 0.3 mg/ml nitro blue tetrazolium and 0.15 mg/ml 5-bromo-4-chloro-3-indolyl phosphate.

### Example 8

### Calmodulin Binding to Fusion Proteins

Soluble and insoluble proteins from the induced and un-induced cultures were separated on 12% SDS-polyacrylamide gels and blotted as described above. To detect the binding of the expressed proteins to ³⁵S-labeled AtCaM, duplicate blots were blocked for 15 min in TBS (50 mM Tris-HCl, pH 7.5, 150 mM NaCl) containing 5 mM of either CaCl₂ or EGTA and 1% nonfat dry milk. The blots were then incubated for 12 h in a mixture of ³⁵S-labeled AtCaM isoforms at 5 mg/ml in the same buffers as above. The filters were then washed with the corresponding buffer and dried before exposing to X-ray film. Binding to biotinylated CaM was detected by blocking the blots in 3% gelatin in TBS/Ca/Mg or TBS/EGTA/Mg (50 mM Tris-HCl, pH 7.5, 50 mM MgCl₂, 150 mM NaCl, and 5 mM of either CaCl₂ or EGTA) at 30° C. The blots were then incubated in 5 mg/ml biotinylated CaM for 2 h in TBS/Ca/Mg or TBS/EGTA/Mg containing 0.1% Tween 20 and 1% gelatin followed by three washes of 10 min each in the corresponding buffers above. The blots were then incubated in Vectastain ABC.HRP in TBS/Ca/Mg or TBS/EGTA/Mg for 30 min, washed 3 times, 10 min each, with the above buffer and the CaM-binding proteins were detected colorimetrically in a substrate solution (0.8 mg/ml diaminobenzidine, 0.4 mg/ml NiCl₂, and 0.009% H₂O₂ in 100 mM Tris-HCl, pH 7.5).

### Example 9

### Preparation of Horseradish Peroxidase (HRP)-labeled CaM and Blot Overlay Assay

HRP was conjugated to CaM and used in blot overlay assays according to the procedure described by Lee et al. (1999) BBA 1433:56-67. Briefly, the Arabidopsis CaM2 was incubated in 25 mM Tris-HCl, pH 7, 2 mM EGTA, and 0.1 M DTT at 55° C. for 1.5 h and the reduced CaM was extensively dialyzed against phosphate-buffered saline at 4° C. Maleimide-activated HRP (Pierce Biotechnology, Inc., Rockford, Ill.) and reduced CaM were incubated at a molar ratio of 1:1 in phosphate-buffered saline at room temperature for 1.5 h. The resulting HRP-CaM complex was verified on a denaturing gel and used in blot-overlay assays. Protein blots were prepared as described above, rinsed in TBST (TBS containing 0.1% (v/v) Tween 20), and blocked by incubating in TBST plus 7% (w/v) non-fat dry milk overnight. The blocked membranes were washed three times, 5 min each, with TBST. After equilibration in the overlay buffer (50 mM imidazole-HCl, pH 7.5, and 150 mM NaCl) for 1 h, the membranes were incubated for 1 h in overlay buffer containing 0.1% gelatin and 1 mg/ml HRP-CaM. The HRP-CaM overlay blots were washed sequentially in three buffers. Wash with each buffer was performed five times for 5 min each. The composition of the buffers was: 1) TBST, 50 mM imidazole-HCl, pH 7.5, 1 mM CaCl₂; 2) 20 mM Tris-HCl, pH 7.5, 0.5% Tween 20, 50 mM imidazole-HCl, pH 7.5, 0.5 M KCl, 1 mM CaCl₂; 3) 20 mM Tris-HCl, pH 7.5, 0.1% Tween 20, 0.5 M KCl, 1 mM MgCl₂. The proteins that bound to HRP-CaM were detected by immersing the blots in a substrate solution as above.

### Example 10

### CaM-binding Studies with Synthetic Peptides

Three synthetic peptides, shown below, were synthesized at the Macromolecular Resource facility at Colorado State University, using standard peptide synthetic methods. The purity of the peptides was about 95%. KCBP refers to kinesin-like calmodulin binding protein.

| | | |
|---|---|---|
| MP-1: | VSKGWRLLALILSAQQRF | (SEQ ID NO:2) |
| MP-2: | AKLDQGSLLRVKA-KLKVAQSSPM | (SEQ ID NO:5) |
| KCBP-peptide: | ISSKEMVRLKKLVAYWKEQAGKK | (SEQ ID NO:6) |

### Example 11

### Slot Blot Analysis

The interaction between synthetic peptides and AtCaM isoforms was analyzed by applying synthetic peptides to a nitrocellulose membrane in a slot blot apparatus, and incubating the membrane individually with ³⁵S-labeled AtCaM isoforms using the method described above.

### Example 12

### Calmodulin Shift Assays

The interaction of CaM with synthetic peptide was also analyzed using electrophoretic mobility shift of CaM in the presence of a synthetic peptide. Each of the AtCaM isoforms and bovine CaM (166 pmol) were incubated with increasing concentrations of maize NPG1-synthetic peptide (166, 332, and 664 pmol) in the presence of 4 M urea, in a buffer containing 100 mM Tris-HCl, pH 7.5, and 1 mM CaCl₂ or 5 mM EGTA at room temperature for 1 h in a total volume of 20 ml. Then, 10 ml of sample buffer (0.375 M Tris-HCl, pH 6.8, 30% glycerol, and 0.023% bromphenol blue) was added to the samples and the mixture was electrophoresed in 12% polyacrylamide gels containing 4 M urea or 7.5% glycerol, 0.375 M Tris-HCl, pH 8.8, and either 1 mM CaCl₂ or 5 mM EGTA. The gels were run at a constant voltage of 25 V per gel in an electrode buffer (25 mM Tris-HCl, pH 8.3, 192 mM glycine, and either 1 mM CaCl₂ or 5 mM EGTA). The gels were stained with 0.25% Coomassie Blue R-250 in 7.5% acetic acid and 50% methanol for 1 h and then destained with 30% methanol and 7% acetic acid.

### Example 13

### Fluorescence Spectroscopy Assay

The tryptophan fluorescence spectra of free and CaM-bound synthetic peptide were recorded with a Hitachi-F-3010/4010 spectrofluorimeter as described in Reddy et al. (1996) supra. NPG1 peptide and AtCaMs (166 pmol each) were mixed in a 600-ml reaction volume. The tryptophan residue in free and CaM-bound synthetic NPG1 peptide was excited at 290 nm and the emission wavelength values were recorded from 300 to 400 nm with a bandwidth of 5 nm in a 5-mm quartz cell at 25° C. Samples were incubated for 1 h at 25° C. prior to spectroscopic measurements. Corrections were made for the protein and solvent blanks.

### Example 14

### Binding of Fusion Proteins to CaM-Sepharose Columns

Bovine CaM Sepharose-4B was obtained from Amersham Pharmacia Biotech UK Ltd. (Buckinghamshire, UK). AtCaM isoforms were conjugated to Sepharose-4B. Briefly 1 g of CNBr-activated Sepharose-4B was rehydrated in 1 mM HCl and then washed with 1 mM HCl on sintered glass for 15 min to remove additives. Ten mg of each of the CaM isoforms 2, 4, and 6 were dialyzed in the coupling buffer (0.1 M NaHCO₃, pH 8.3, 0.5 M NaCl) and incubated with the washed Sepharose in 5 ml of coupling buffer. The mixture was rotated at room temperature for 2 h in a 15-ml tube after which it was centrifuged at low speed (1000×g) and washed 3 times with 5 gel volumes of coupling buffer to remove unconjugated ligand. After centrifugation, the washing solution was replaced with blocking solution (0.1 M Tris-HCl, pH 8.0) and incubated for 2 h at room temperature to block remaining active groups. The beads were then washed three times, each time with 0.1 M acetate buffer containing 0.5 M NaCl followed by 0.1 M Tris-HCl, pH 8.0, containing 0.5 M NaCl. The solution was then replaced by CaM-Sepharose binding buffer (50 mM Tris-HCl, pH 7.5, 150 mM NaCl, and 5 mM CaCl₂) and the slurry was degassed and packed into a column. Insoluble fusion protein from P3 clone was dissolved in binding buffer containing 6 M urea. To purify these proteins on CaM-Sepharose column, the column was equilibrated in the binding buffer with 6 M urea and the dissolved proteins were loaded. The unbound protein was washed with the same buffer, and the bound protein was eluted with binding buffer containing 6 M urea except that CaCl₂ was replaced with 7 mM EGTA. The fractions were analyzed on 12% SDS-polyacrylamide gels and detected with T7-AP, biotinylated CaM, or ³⁵S-labeled AtCaM in the presence or absence of CaCl₂.

### Example 15

### Northern Blot Analysis

Total RNA was extracted from maize pollen and kernels as according to Broadwater and Bedinger (1994) In: The maize Handbook, Freeling, M. and Walbot, V. eds. Pp. 538-540, Springer-Verlag, N.Y. Pollen was ground in liquid nitrogen and sand, then homogenized in 3 volumes of lysis buffer (50 mM EGTA, 100 mM NaCl, 1% SDS, 100 mM Tris-HCl, pH 7.6, 50 mM β-mercaptoethanol) containing 10 mM vanadyl ribonucleoside. The homogenate was extracted several times with an equal volume of phenol/chloroform and centrifuged for 10 min at 10,000 rpm until there was no detectable interphase. RNA was precipitated with sodium acetate and ethanol, dissolved in diethyl pyrocarbonate-treated water, and reprecipitated twice with equal volumes of 5 M LiCl on ice for 1 h. RNA from other maize tissues was extracted using the Trizol method according to manufacturer's instructions (Gibco BRL, Gaithersburg, Md.). RNA was loaded on denaturing 1% agarose gels containing formaldehyde and blotted and probed according to standard procedures.

### Example 16

### Southern Blot Analysis

Maize genomic DNA was digested with different restriction enzymes, electrophoresed in a 0.8% agarose gel, and transferred onto a Hybond nylon membrane (Amersham Pharmacia Biotech UK Ltd., Little Chalfont, Buckinghamshire, UK). The DNA was fixed to the membrane by UV cross-linking. The blot was hybridized to the radiolabeled 1.2 kb cDNA at 65° C. and washed under high stringency conditions as described in Sambrook et al. (1989) supra.

### Example 17

### Antibody Production

About 150 mg of fusion protein induced from the 1.2 kb cDNA (P3) clone and purified on a CaM-Sepharose column was electrophoresed on a preparative SDS-polyacrylamide mini-gel. The gel was washed 3 times with distilled water, stained in water-based 0.05% Coomassie R-250 for 30 min, and rinsed in several changes of water for 1 h. The protein band (32 kDa) was cut, rinsed briefly with 50 ml of water, and then overnight with 5 ml of buffer (50 mM Tris-HCl, pH 7.5, at 4° C.). The gel pieces were solubilized, mixed with Freund's incomplete adjuvant (1:1 ratio), and injected intradermally at multiple spots into New Zealand White rabbits. Booster injections were performed on 14, 28, 42, and 56 days after initial injection. Bleeds were performed before the initial injection and on the day of each booster injection. The terminal bleed was collected on day 140. Serum from day 126-bleed was used for antibody purification and immunodetection studies. Anti-NPG1 antibodies were affinity purified using a modified method of the Millipore technical protocol TP015 (Millipore Corp., Bedford, Mass.). About 1 mg of the purified NPG1 protein was electrophoresed on an SDS-polyacrylamide gel and transblotted onto a polyvinylidene difluoride membrane. The membrane was stained briefly with Ponceau stain (0.1% (w/v) in 1% (v/v) acetic acid), destained in distilled water, and a strip was cut with the band corresponding to NPG1 protein. The strip was cut into 1×2-cm pieces, equilibrated in 0.5 M potassium phosphate buffer, pH 7.4, rinsed in phosphate-buffered saline containing 0.1% Tween 20, and then incubated in 10% monoethanolamine in 1 M NaHCO₃ for 2 h. Following two 30-min rinses in phosphate-buffered saline containing 0.1% Tween 20, the strips were incubated with 2 ml of serum for 3.5 h. The strips were rinsed three times and incubated in 0.9 ml of 100 mM glycine, pH 2.5, for 10 min. The solution was removed and neutralized with 0.1 ml of 1 M Tris, pH 8.0 (Harlow and Lane (1988) supra).

### Example 18

### Immunodetection of Maize NPG1 Polypeptide

Proteins from pollen grains, germinating pollen grains, and other maize tissues were extracted by grinding tissues in liquid nitrogen and homogenizing in extraction buffer (50 mM Tris-HCl, pH 7.4, 5 mM EDTA, 250 mM sucrose, 5 mM DTT, and complete protease inhibitor mixture). After centrifuging at 14,000×g for 20 min, the supernatant was collected and used for electrophoresis. Microsomal and soluble fractions of the pollen extract were separated by centrifuging at 100,000×g, and washing the pellet twice with extraction buffer. The microsomal fraction was dissolved in sample buffer. Proteins were separated on an SDS-polyacrylamide gel and transblotted onto nitrocellulose membrane using a Bio-Rad transfer cell. After blocking with 3% gelatin in antibody buffer (20 mM Tris-HCl, pH 7.5, 0.5 M NaCl) for 2 h, the membranes were incubated with the affinity-purified NPG1 antibody (1:5,000) in antibody buffer containing 1% gelatin for 2 h at 30° C. The membrane was then washed with antibody buffer containing 0.05% Tween 20 followed by incubation for 1 h at 30° C. in 1:4000 dilution of goat anti-rabbit IgG conjugated to alkaline phosphatase (Stratagene, La Jolla, Calif.). Immunoreactive bands were detected colorimetrically by immersing the filter in substrate solution (0.3 mg/ml nitro blue tetrazolium and 0.15 mg/ml 5-bromo-4-chloro-3-indolyl phosphate in AP buffer).

### Example 19

### Purification of Native NPG1 Polypeptide from Maize Pollen Extract Using a CaM-Sepharose Column

Maize pollen proteins were extracted in a buffer containing 50 mM Tris-HCl, pH 7.4, 250 mM sucrose, 5 mM DTT, and complete protease inhibitor mixture. The extract was first centrifuged at 14,000×g for 20 min and then at 100,000×g for 15 min at 4° C. to obtain the supernatant containing soluble proteins. Prior to loading the supernatant onto the CaM-Sepharose column (Amersham Pharmacia Biotech UK Ltd., Little Chalfont, Buckinghamshire, UK) the concentration of Ca²⁺ was adjusted to 1.25 mM. The flow-through was collected and saved. The column was washed thoroughly with binding buffer (50 mM Tris-HCl, pH 7.5, 150 mM NaCl, 1.25 mM CaCl₂) to remove unbound proteins. Washes equivalent to 1-bed volume of the column were collected and saved. The CaM-binding proteins were eluted with elution buffer (50 mM Tris-HCl, pH 7.5, 150 mM NaCl, 5 mM EGTA). The absorbance of the eluted fractions was recorded at 235 and 280 nm to compare the elution profile of protein with EGTA. Initial soluble extract, flow-through, wash fraction, and eluted protein were separated on three denaturing gels. One gel was stained with Coomassie Blue and the other two were blotted onto nitrocellulose membranes and probed with NPG1-specific antibody or HRP-CaM.

### Example 20

### Pull-down Assay with CaM-Sepharose Beads

Five-hundred ml of pollen soluble proteins prepared as described above were mixed and incubated with 0.2 ml of CaM-Sepharose beads at room temperature for 30 min. After a brief centrifugation, the supernatant (designated as flow-through) was collected and saved. The beads were washed three times with the binding buffer. The first 0.5 ml of wash was saved. The bound proteins were eluted with the elution buffer. The flow-through, wash, and eluted proteins were separated on three SDS gels and processed as above.

### Example 21

### Cloning of Arabidopsis NPG1 cDNA

DNAse treated RNA from pollen was used to synthesize first strand cDNA with an oligo (dT) primer. Primers corresponding to 5′ (5′-ATGCTCGGGAATCAATCCGCGG-3′, SEQ ID NO:13) and 3′ (5′-TTAAAGAATGGTTGAGAAGCTTTC-3′, SEQ ID NO:14) ends of NPG1 were designed based on NPG1 gene (At2g43040) sequence in the TAIR database. PCR was performed in a final volume of 50 μL using the TaKaRa Ex Taq PCR system according to the manufacturer's instructions (Takara Bio. Inc., Shiga, Japan). The amplified product was resolved by electrophoresis, gel purified, cloned into pGEM-T Easy vector (Promega, Madison, Wis.), and sequenced.

### Example 22

### Bacterial Expression and Detection of Arabidopsis NPG1 Polypeptide

A 2.0 kb EcoRI fragment of AtNPG1 cDNA containing the entire coding region was subcloned into pET28a and the orientation of the cDNA was confirmed by restriction analysis and sequencing. Induction of fusion protein, its purification on CaM Sepharose column and detection with horseradish peroxidase CaM isoforms was performed essentially as described earlier (Reddy and Reddy (2000) J. Biol. Chem. 275:35457-35470).

### Example 23

### Expression Analysis of Arabidopsis NPG1

Arabidopsis pollen was collected according to Huang et al.(1997) Plant Mol. Biol. 33:125-139. RNA from pollen and tissues was isolated using the TRIzol reagent according to the protocol provided by Gibco BRL (Gaithersburg, Md.) with the following modifications for pollen RNA extraction. Five hundred μl of TRIzol reagent plus 300 μl of glass beads (710-1180 μM) were added to 200 μl of pollen and vortexed continuously at a high speed for 15 min. and then another 500 μl of TRIzol reagent was added. RNA pellets were dissolved in either deionized formamide (for RNA gel blots) or RNAse-free water (for RT-PCR). Fifty micrograms of total RNA from each tissue was electrophoresed, blotted, hybridized with ³²P-labeled probe and washed under high stringency conditions. Amplification of NPG1 transcript by RT-PCR was as described above.

### Example 24

### Screening of T-DNA Insertion Lines for Arabidopsis NPG1 Mutant

A total of 60,400 T-DNA-tagged Arabidopsis mutants were screened that were generated at the University of Wisconsin Arabidopsis knockout facility using an NPG1-specific forward (5′ACCAAAGAGGGAATTTAGAAGGCGCACTT-3′, SEQ ID NO:15) or reverse (5′-TACGTAACTCCTAGCTAGGTTGTTTGGCT-3′, SEQ ID NO:16) primer together with T-DNA left or right border primer (left border primer, 5′-CATTTTATAATAACGCTGCGGACATCTAC-3′ and right border primer, 5′-TGGGAAAACCTGGCGTTACCCAACTTAAT-3′, SEQ ID NOS:17 and 18 respectively). Screening of superpools and pools was performed at the Arabidopsis Knockout Facility. By Southern analysis of PCR-amplified products we identified a T-DNA insertion in NPG1 with one primer combination (T-DNA left border and NPG1 reverse primers). We then reamplified the hybridizing band and sequenced the product to confirm that the insertion was in the coding region of NPG1. Final screening and identification of NPG1 knockout line was performed at Colorado State University. To determine if the mutated gene is transmitted through pollen we pollinated a male sterile (cer6-2) mutant with pollen from NPG1/npg1 mutant. Seeds from this cross were germinated on plates with or without kanamycin.

### Example 25

### Generation of NPG1/npg1, qrt/qrt Double Mutant

Tetrad analysis was performed by generating a double mutant. The pollen from qrt/qrt mutant (quartet1) (Preuss et al. (1994) Science 264:1458-1460) was used to pollinate emasculated flowers of NPG1/npg1 mutant. We selected the progeny on kanamycin plates for NPG1/npg1, QRT/qrt and selfed. Seeds from selfing were grown on kanamycin plates and plants that showed qrt phenotype (NPG1/npg1, qrt/qrt—one out of every four kanamycin resistant plants) were selected.

### Example 26

### Microprojectile Bombardment of Pollen

The helium-driven PDS-1000/He particle delivery system (Bio-Rad Laboratories, Hercules, Calif.) was used for the biolistic transformation of mid-bicellular grains of tobacco pollen (Nicotiana tabaccum). Tobacco pollen was resuspended to 1×10⁶ cells in 50-100 μl of germination medium (0.1 mg/ml H₃BO₃, 0.71 mg/ml Ca(NO₃)₂ 4H₂O, 0.2 mg/ml MgSO₄ 7H₂O, 0.1 mg/ml KNO₃, 10% Sucrose, 0.5 mg/ml MES, 1.0 mg/ml casein hydrolysate, pH 5.9) and placed in the middle of a 5 cm petri dish previously rinsed with the same buffer to create an even monolayer of cells. Each pollen-monolayer was bombarded at least 3 times as described earlier (Ottenschlager et al. (1999) Transgenic Res. 8:279-294) and 5 ml germination medium was added. The plates were then left in the dark without shaking for 12-24 hours in a humid chamber at 21±3° C. Green fluorescent proteins (GFP) expressing pollen were initially scored on the plates and later transferred onto a glass slide for confocal microscopy.

### Example 27

### Pollen Germination

In vitro germination of Arabidopsis pollen was performed in acid-washed depression slides in liquid medium essentially as described in Fan et al. (2001) J. Exp. Bot. 52:1603-1614. About 60 to 70% of pollen germinated in this medium. Pollen from qrt/qrt or NPG1/npg1, qrt/qrt flowers stage 12 or 13 (Smyth et al. (1990) Plant Cell 2:755-767) was carefully tapped onto the slides and the medium was added. Slides were then incubated in a humid chamber at 21±3° C. without light for 10-24 hrs.

### Example 28

### Generation and Analysis of Transgenic Plants

The following constructs were used for either transient and/or stable expression. A 2.4 kb fragment (NP, NPG1 Promoter) upstream of the translation initiation codon of the NPG1 gene was amplified with forward (5′-CATGCCATGGTATGAGTCGAGTGTCTGACTT-3′, SEQ ID NO:11) and reverse 5′-TCGCCATGGTTCTTCACCTTTTAGACTA-3′, SEQ ID NO:12) primers carrying a NcoI site (underlined). The PCR fragment was cloned into pGEM-TE vector, sequenced and used to replace the 35S promoter region (NcoI-NcoI) in pBA002-GFP binary vector to generate NP<GFP>Nos. For complementation of the NPG1 mutant, another construct was made by replacing the GFP fragment (NcoI-SmaI) with a blunted SpeI-NcoI fragment of NPG1 cDNA in pBA002 based construct NP<GFP>Nos to create NP<NPG1>Nos. The constructs were verified by sequencing and introduced into Agrobacterium GV3 101 strain and used to transform Arabidopsis plants using the floral-dip method. Wild type plants were transformed with NP<GFP>Nos and NPG1/npg1 plants were transformed with NP<NPG1>Nos. Seeds from infiltrated plants were collected, surface-sterilized, stratified and selected on appropriate selection plates.

### Example 29

### Histology, Light Microscopy and Confocal Laser Scanning Microscopy

Inflorescences were harvested, dehydrated in a standard ethanol series, fixed (formamide and glutaraldehyde solutions) and embedded in Paraplast tissue medium (Ruzin, S. E. (1999) Plant Microtechnique and Microscopy (Oxford University Press, Oxford)). Samples were sectioned and mounted on poly-L-lysine-coated glass slides and then stained. Tissue sections were observed with a Leitz Laborlux S microscope. Images were captured using Kodak Digital Science DC-120 zoom camera. For nuclei staining 1 μl of DAPI (4′, 6-diamidino-2 phenylindole) solution (1 mg/ml) was added to 1-2 ml of mounting buffer (0.1M Tris-HCl, pH9.0; 50% glycerol) and the pollen were examined in UV-light after 10-30 minutes. GFP images were captured using a fluorescence microscope with an integrated confocal imaging (FVX-IHRT Fluoview Confocal LSM) system from Olympus (Melville, N.Y.). Images were acquired using the Fluoview software provided by the manufacturer. Scanning electron microscopy was performed essentially as described earlier (Preuss et al. (1993) supra). All images were processed with Adobe PhotoShop 5.0 software.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

We claim:

1. An isolated nucleic acid molecule encoding a NPG1 (No Pollen Germination1) polypeptide having calmodulin binding activity, wherein said NPG1 polypeptide comprises an amino acid sequence at least 60% identical to the amino acid sequence of SEQ ID NO:1 or 10.

2. The isolated nucleic acid molecule of claim 1, wherein said NPG1 polypeptide comprises the amino acid sequence as shown in SEQ ID NO:1.

3. The isolated nucleic acid molecule of claim 1 wherein said NPG1 polypeptide comprises the amino acid sequence as shown in SEQ ID NO:10.

4. The isolated nucleic acid molecule of claim 2, wherein said NPG1 polypeptide is encoded by the nucleotide coding sequence as shown in SEQ ID NO: 7.

5. The isolated nucleic acid molecule of claim 3, wherein said NPG1 polypeptide is encoded by the nucleotide coding sequence as shown in SEQ ID NO: 9.

6. An isolated DNA fragment capable of directing pollen specific expression of a nucleic acid molecule operably linked thereto, wherein said DNA fragment comprises the nucleotide sequence as shown in SEQ ID NO:3 or 4.

7. An isolated polypeptide capable of binding calmodulin in a Ca⁺⁺ dependent manner and is expressed specifically in pollen, wherein said polypeptide comprises the amino acid sequence as shown in SEQ ID NO: 1 or 10.

8. The isolated peptide of claim 7, wherein said peptide comprises the amino acid sequence of SEQ ID NO:1, amino acid residues 418-474.

9. The isolated peptide of claim 8, wherein said peptide comprises the amino acid sequence of SEQ ID NO:1, amino acid residues 421-438.

10. The isolated peptide of claim 7, wherein said peptide comprises the amino acid sequence of SEQ ID NO:10, amino acid residues 464-481.

11. An expression vector comprising the nucleic acid molecule of claim 1, wherein a coding sequence of said molecule is operably linked to and expressed under control of transcription and translation regulatory elements.

12. The expression vector of claim 11 wherein said coding sequence encodes a polypeptide comprising the amino acid sequence as given in SEQ ID NO:1 or 10.

13. The expression vector of claim 11 wherein said vector is selected from the group consisting of bacterial, mammalian, baculovirus, and yeast vector.

14. A recombinant host cell, wherein said cell comprises the expression vector of claim 11.

15. A method for producing a recombinant NPG1 protein, said method comprising the steps of:
(a) introducing the expression vector of claim 11 into a host cell selected from the group consisting of bacterial cell, yeast cell, mammalian cell, and insect cell; and
(b) culturing under conditions the recombinant NPG1 protein is produced,
whereby said recombinant NPG1 protein binds calmodulin in a Ca²⁺dependent manner.

16. A purified antibody that binds specifically to NPG1 protein the amino acid sequence comprising the amino acid sequence of SEQ ID NO:1 or 10.

17. The purified antibody of claim 16, wherein said antibody selectively binds to an epitope in the peptide of claim 9.

18. The purified antibody of claim 16, wherein said antibody selectively binds to an epitope in the peptide of claim 10.

19. An expression vector for expressing a gene of interest specifically in pollen comprising a pollen specific regulatory element isolated from maize or Arabidopsis, operably linked to a DNA fragment encoding a gene of interest.

20. The expression vector of claim 19, wherein said pollen specific elements comprise the nucleotide sequence as shown in SEQ ID NO:3 or 4.

21. The expression vector of claim 19, wherein said DNA fragment comprises of at least 10 consecutive nucleotides complementary to the nucleotide sequence as shown in SEQ ID NO:7 or 9.

22. The expression vector of claim 19, wherein said DNA fragment comprises of at least 10 consecutive nucleotides complementary to a nucleotide sequence encoding an allergen.

23. A single-stranded nucleic acid that hybridizes with a nucleotide sequence encoding the amino acid sequence as shown in SEQ ID NO:1 or 10 under wash conditions of room temperature, 2×SSC, and 0.5% SDS; then wash conditions of 65° C., 0.1× SSC, and 0.1%SDS.

24. An antisense oligonucleotide that causes male sterility in plants, wherein said antisense oligonucleotide comprises the nucleotide sequence complementary to the nucleotide sequence as shown in SEQ ID NO:7 or 9.

25. A method of expressing a nucleic acid molecule specifically in pollen comprising the steps of:
(a) preparing an expression vector comprising a pollen specific regulatory elements operably linked to a nucleic acid molecule encoding a gene of interest or complementary to a coding sequence encoding a gene of interest
(b) introducing said expression vector into a plant cell
(c) selecting the plant cell containing the expression vector
(d) regenerating a transgenic plant from the selected cells
whereby the transgenic plant expresses the nucleic acid molecule or a gene product encoded by the coding sequence.

26. The method of claim 25 wherein the pollen specific regulatory elements comprise the nucleotide sequence as shown in SEQ ID NO:3 or 4.

27. The method of claim 25, wherein said coding sequence encodes maize NPG1.

28. The method of claim 25, wherein said coding sequence encodes an allegen.

29. A method for controlling male fertility in a plant by modulating expression of NPG1 polypeptide wherein said modulation is achieved by introducing an antisense nucleotide that is complementary to the sense strand of the NPG1 gene.

30. The method of claim 29 wherein said plant is maize.

31. A method for producing maize hybrid seed, comprising the steps of:
(a) planting in cross-pollinating juxtaposition, a first seed from a selected male fertile parent line and a second seed selected from a female parent line having male sterility resulting from the modification of the NPG1 polypeptide
(b) growing the seed to mature plants and cross-pollinating the male-sterile female plant with pollen from the male fertile plant; and
(c) harvesting the seed from the male sterile female plant.

32. A method of generating male sterile plants, said method comprising the steps of:
(a) transforming a cell or group of cells of said plant with an expression vector comprising regulatory elements capable of directing expression of a nucleotide sequence when operably linked downstream thereof in pollen tissue, wherein said regulatory elements comprise a nucleotide sequence set forth in SEQ ID NO:3 or 4 and wherein said regulatory elements direct expression of a nucleotide sequence having a deleterious effect on pollen germination; and
(b) regenerating a transgenic plant from said transformed cells and growing or maintaining said transgenic plant under conditions having a deleterious effect on said pollen germination resulting said plant being male sterile, wherein the nucleotide sequence having a deleterious effect is antisense to the nucleotide as shown in SEQ ID NO:7 or 9.

33. A transgenic plant comprising an expression vector, said expression vector capable of down regulating expression of endogenous NPG1 gene such that said transgenic plant is male sterile.

34. The transgenic plant of claim 33 wherein said plant is maize.

35. A method for isolating calmodulin polypeptide or fragments thereof comprising the steps of:
(a) presenting the NPG1 polypeptide or fragment thereof to a cell extract suspected of containing calmodulin polypeptide in the presence of Ca²⁺
(b) separating NPG1-calmodulin complex from the cell extract; and
(c) separating calmodulin from the complex by adding EGTA
whereby purified calmodulin polypeptide is obtained.

36. A method of identifying a protein or peptide that interacts with the NPG1 polypeptide comprising the steps of:
(a) preparing an expression library from the RNA preparation extracted from pollen
(b) screening the library with a labeled NPG1 polypeptide
(c) isolating a clone in the library that expresses the protein or peptide that binds to the labeled NPG1 polypeptide
(d) identifying the protein or peptide by sequencing the nucleotide sequence of the clone.
